SEQUENCE LISTING (7 sequences)

SEQ ID NO: 1 - DNA, 21 bp, Artificial
  synthetic shRNA targeting PHF8
gcttcatgatcgagtgtgaca

SEQ ID NO: 2 - DNA, 19 bp, Artificial
  synthetic shRNA targeting AF4p12
gcaggaatgtgctcagtat

SEQ ID NO: 3 - DNA, 19 bp, Artificial
  synthetic shRNA targeting AF4p12
ggctgtttcagacaattca

SEQ ID NO: 4 - DNA, 19 bp, Artificial
  synthetic shRNA targeting LSD1
gaaggctcttctagcaata

SEQ ID NO: 5 - DNA, 19 bp, Artificial
  synthetic shRNA targeting LSD1
gcaccttataacagtgata

SEQ ID NO: 6 - DNA, 19 bp, Artificial
  synthetic shRNA targeting BRG1
cgacgtacgagtacatcat

SEQ ID NO: 7 - DNA, 19 bp, Artificial
  synthetic shRNA targeting BRG1
gggtaccctcaggacaaca

(19) United States
(12) Patent Application Publication
(10) Pub. No.: US 2015/0276760 A1
(43) Pub. Date: Oct. 1, 2015

(54) Method for Screening a Compound Capable of Inhibiting the Notch1 Transcriptional Activity

(71) Applicants: INSERM (institut National de la Sante et de la Recherche Medicate), Paris (FR); Centre National de la Recherche Scientifique-CNRS, Paris (FR); Universite Paris-Est Creteil Val de Mame, Centeil Cedex (FR); Assistance Publique-Hopitaux de Paris, Paris (FR)

(72) Inventors: Yves Levy, Creteil (FR); Monsef Benkirane, Montpellier Cedex 5 (FR); Ahmad Yatim, Creteil (FR)

(21) Appl. No.: 14/433,426

(22) PCT Filed: Oct. 4, 2012
(86) PCT No.: PCT/IB2012/002303

§ 371 Date: Apr. 3, 2015

(51) Int. Cl. G01N 33/68; A61K 31/135; A61K 31/7105; A61K 31/5513

(52) U.S. Cl. CPC G01N 33/6872; A61K 31/5513; A61K 31/135; A61K 31/7105; G01N 2333/705; G01N 2500/04; G01N 2500/10

(57) ABSTRACT

The present invention relates to a method for screening a compound capable of inhibiting the Notch1 transcriptional activity. The present invention also relates to compounds useful in the prevention or treatment of cell proliferative diseases and disorders associated with overexpression and/or activation of Notch1.

### FIELD OF THE INVENTION

The present invention describes a direct interaction between the intracellular active form of NOTCH1 (ICN1) and several nuclear proteins forming a multifunctional complex and regulating the Notch1 transcriptional activity. The present invention relates to a method for screening a compound capable of inhibiting the Notch1 transcriptional activity. The present invention also relates to compounds useful in the prevention or treatment of cell proliferative diseases and disorders associated with overexpression and/or activation of Notch1.

### BACKGROUND OF THE INVENTION

Notch pathway signaling is involved in numerous cellular processes, including cell fate determination, differentiation, proliferation, apoptosis, migration and angiogenesis. In mammals, there are four Notch proteins (sometimes called “Notch receptors”), designated Notch1-Notch4. All four Notch proteins have a similar domain structure, which includes an extracellular domain, a negative regulatory (NRR) domain, a single-pass transmembrane domain, and an intracellular domain. The extracellular domain contains a series of EGF-like repeats that are involved in ligand binding. During maturation, the Notch polypeptide is cleaved by a furin-like protease. This cleavage divides the Notch protein into two subunits that are held together by the NRR. In the absence of ligand binding, the NRR domain functions to keep the Notch protein in a protease-resistant conformation. The intracellular domain is a transcription factor called the intracellular domain of Notch (ICN), which is released upon proteolytic cleavage by gamma secretase, in response to binding of the Notch protein by a ligand. In mammals, the Notch ligands are Delta-like and Jagged. When the ICN is released, it travels to the nucleus, where it activates transcription of the Notch-responsive genes, HES1, HESS, NRARP, Deltex1 and c-MYC.

While Notch proteins play crucial roles in normal development, dysregulation of the Notch proteins is associated with various types of cancer, including T-cell acute lymphoblastic leukemia (T-ALL), breast cancer, colon cancer, ovarian cancer and lung cancer (Miele et al, 2006). Indeed, abnormal expression or mutations in the different components of the pathway are associated with a number of diseases and cancers. An enhanced activity of Notch signalling resulting from a mutation in the extracellular domain is implicated in the progression of T-ALL. Several therapeutic agents have been developed to target the Notch signalling pathway such as, γ-secretase inhibitors and antibodies targeting different regions of the Notch receptor (e.g. antibodies targeting the NRR domain). For instance, one therapeutic approach for the treatment of cancer is inhibition of Notch pathway signaling. Inhibition of Notch pathway signaling has been achieved using monoclonal antibodies (Wu et al, 2010).

However, the current inhibitors have their own disadvantages including lack of selectivity. It results that a more selective approach to target downstream protein-protein interactions (ppi) would provide an attractive approach to the design of new therapeutic agents that target this pathway since ppi may provide an opportunity to develop selective inhibitors by inhibiting the formation of the transcription complex ICN.

Thus, over the past decades important progress has been made in deciphering Notch signal transduction and identifying processes that are influenced by Notch (Kopan and Ilagan, 2009). The emerging picture posits that most Notch-dependent physiological and pathological processes rely on the ability of nuclear ICN to convert the DNA-binding protein CSL (also known as RBPJ) from a transcriptional repressor into an activator. This regulation involves the formation of a stable ternary complex composed of CSL, ICN and Mastermind-like family of coactivators (MAML) (Nam et al., 2006; Wilson and Kovall, 2006). Although little is known about its physical partners, the CSL-ICN-MAML complex is thought to serve as a platform for recruitment of coactivators and subsequent transcriptional activation to Notch-target genes (Borggrefe and Oswald, 2009). Deciphering how the CSL-ICN-MAML ternary complex orchestrates transcriptional activation and how diversity in the transcriptional program is established depending on the cellular context is a major challenge in the field.

### SUMMARY OF THE INVENTION

In a first aspect, the present invention provides a method for screening a compound capable of inhibiting the Notch1 transcriptional activity, comprising the step consisting of:
(a) identifying a compound that inhibits the specific interaction of intracellular domain of NOTCH1 (ICN1) with a nuclear protein required for Notch1 transcriptional activity as depicted in Table 1, or
(b) identifying a compound that inhibits the expression of a nuclear protein as depicted in Table 1, or
(c) identifying a compound that inhibits the activity of a nuclear protein as depicted in Table 1.

In a second aspect, the present invention relates to a compound that inhibits the interaction between ICN1 and a nuclear protein required for Notch1 transcriptional activity as depicted in Table 1 for use in the prevention or treatment of cell proliferative diseases and disorders associated with overexpression and/or activation of Notch1.

In a third aspect, the present invention further relates to a compound that inhibits the activity of PHF8 or AF4p12 for use in the prevention or treatment of cell proliferative diseases and disorders associated with overexpression and/or activation of Notch1.

### DETAILED DESCRIPTION OF THE INVENTION

Using tandem affinity chromatography, followed by mass spectrometry, the inventors purified the intracellular active form of NOTCH1 (ICN1) and identified its associated partners in human T-ALL cells. The inventors found a large set of proteins associated with ICN1, including transcriptional regulators and protein modifiers. Moreover, the inventors found that ICN1 associates with lineage-specific transcription factors and components of other signaling pathways that could cooperate with Notch to confer a specific program of gene expression. Importantly, biochemical and functional analysis led to the identification of several new components of the Notch-activation complex and provide new insights into the molecular mechanisms that govern Notch-mediated activation of its target genes.

Accordingly, the present invention relates to the identification of nuclear proteins that interact with ICN required for Notch1 activation, and high throughput assays to identify substances that interfere with the specific interaction between ICN and nuclear proteins. Interfering substances that inhibit Notch1 activation can be used therapeutically to treat cell proliferative diseases and disorders, including certain forms of cancer, associated with overexpression and/or activation of Notch1.

All the human proteins such as ICN1 and nuclear proteins defined in the Table 1 are known per se by the skilled man in the art.

The present invention describes various biological assays that may be used for screening substances that can inhibit the Notch1 transcriptional activity.

### Screening Methods for Identifying a Compound Capable of Inhibiting the Notch1 Transcriptional Activity

Accordingly, the present invention method for screening a compound capable of inhibiting the Notch1 transcriptional activity, comprising the step consisting of:
(a) identifying a compound that inhibits the specific interaction of intracellular domain of NOTCH1 (ICN1) with a nuclear protein required for Notch1 transcriptional activity as depicted in Table 1, or
(b) identifying a compound that inhibits the expression of a nuclear protein as depicted in Table 1, or
(c) identifying a compound that inhibits the activity of a nuclear protein as depicted in Table 1.

Biological Assays for Identifying a Compound that Inhibits the Specific Interaction of the Intracellular Domain of NOTCH1 (ICN1) with a Nuclear Protein Required for Notch1 Transcriptional Activity:

A particular aspect of the invention relates to an assay for identifying a compound that inhibits the specific interaction of intracellular domain of NOTCH1 (ICN1) with a nuclear protein required for Notch1 transcriptional activity as depicted in Table 1 comprising:

(a) contacting a protein or peptide containing an amino acid sequence corresponding to the binding site of the intracellular domain of NOTCH1 (ICN1) with a protein or peptide having an amino acid sequence corresponding to the binding site of the nuclear protein, under conditions and for a time sufficient to permit binding and the formation of a complex, in the presence of a test compound, and

(b) detecting the formation of a complex, in which the ability of the test compound to inhibits the interaction between the ICN1 protein and the nuclear protein is indicated by a decrease in complex formation as compared to the amount of complex formed in the absence of the test compound.

The term “protein” means herein a polymer of amino acids having no specific length. Thus, peptides, oligopeptides and polypeptides are included in the definition of “proteins” and these terms are used interchangeably throughout the specification, as well as in the claims. The term “protein” does not exclude post-translational modifications that include but are not limited to phosphorylation, acetylation, glycosylation and the like. Also encompassed by this definition of “protein” are homologs thereof.

Proteins of the invention may be produced by any technique known per se in the art, such as without limitation, any chemical, biological, genetic or enzymatic technique, either alone or in combination(s). Knowing the amino acid sequence of the desired sequence, one skilled in the art can readily produce said protein, by standard techniques for production of proteins. For instance, they can be synthesized using well-known solid phase method, preferably using a commercially available peptide synthesis apparatus (such as that made by Applied Biosystems, Foster City, Calif.) and following the manufacturer's instructions.

Alternatively, the proteins of the invention can be synthesized by recombinant DNA techniques as is now well-known in the art. For example, these fragments can be obtained as DNA expression products after incorporation of DNA sequences encoding the desired (poly)peptide into expression vectors and introduction of such vectors into suitable eukaryotic or prokaryotic hosts that will express the desired protein, from which they can be later isolated using well-known techniques.

Accordingly, the assay as above described may be useful for identifying compounds that inhibit the specific interaction of the ICN1 protein with the nuclear protein.

In one embodiment the step b) consists in generating physical values which illustrate or not the ability of said test compound to inhibits the interaction between said ICN1 protein and said nuclear protein and comparing said values with standard physical values obtained in the same assay performed in the absence of the said test compound. The “physical values” that are referred to above may be of various kinds depending of the binding assay that is performed, but notably encompass light absorbance values, radioactive signals and intensity value of fluorescence signal. If after the comparison of the physical values with the standard physical values, it is determined that the said test compound inhibits the binding between said ICN1 protein and said nuclear protein, then the candidate is positively selected at step b).

The compounds that inhibit the interaction between the ICN1 protein and nuclear protein encompass those compounds that bind either to ICN1 protein or to nuclear protein, provided that the binding of said compounds of interest then prevents the interaction between ICN1 protein and nuclear protein.

### Labelled Polypeptides

In one embodiment, any protein or peptide of the invention is labelled with a detectable molecule for the screening purposes.

According to the invention, said detectable molecule may consist of any substance or substance that is detectable by spectroscopic, photochemical, biochemical, immunochemical or chemical means. For example, useful detectable molecules include radioactive substance (including those comprising ³²P, ²⁵S, ³H, or ¹²⁵I), fluorescent dyes (including 5-bromodesosyrudin, fluorescein, acetylaminofluorene or digoxigenin), fluorescent proteins (including GFPs and YFPs), or detectable proteins or peptides (including biotin, polyhistidine tails or other antigen tags like the HA antigen, the FLAG antigen, the c-myc antigen and the DNP antigen).

According to the invention, the detectable molecule is located at, or bound to, an amino acid residue located outside the said amino acid sequence of interest, in order to minimise or prevent any artefact for the binding between said polypeptides or between the test compound and or any of said polypeptides.

In another particular embodiment, the proteins of the invention are fused with a GST tag (Glutathione S-transferase). In this embodiment, the GST moiety of the said fusion protein may be used as detectable molecule. In the said fusion protein, the GST may be located either at the N-terminal end or at the C-terminal end. The GST detectable molecule may be detected when it is subsequently brought into contact with an anti-GST antibody, including with a labelled anti-GST antibody. Anti-GST antibodies labelled with various detectable molecules are easily commercially available.

In another particular embodiment, the proteins of the invention are fused with a poly-histidine tag. Said poly-histidine tag usually comprises at least four consecutive hisitidine residues and generally at least six consecutive histidine residues. Such a polypeptide tag may also comprise up to 20 consecutive histidine residues. Said poly-histidine tag may be located either at the N-terminal end or at the C-terminal end. In this embodiment, the poly-histidine tag may be detected when it is subsequently brought into contact with an anti-poly-histidine antibody, including with a labelled anti-poly-histidine antibody. Anti-poly-histidine antibodies labelled with various detectable molecules are easily commercially available.

In a further embodiment, the proteins of the invention are fused with a protein moiety consisting of either the DNA binding domain or the activator domain of a transcription factor. Said protein moiety domain of transcription may be located either at the N-terminal end or at the C-terminal end. Such a DNA binding domain may consist of the well-known DNA binding domain of LexA protein originating form E. Coli. Moreover said activator domain of a transcription factor may consist of the activator domain of the well-known Gal4 protein originating from yeast.

### Two-Hybrid Assay

In one embodiment of the assay according to the invention, the proteins of the invention comprise a portion of a transcription factor. In said assay, the binding together of the first and second portions generates a functional transcription factor that binds to a specific regulatory DNA sequence, which in turn induces expression of a reporter DNA sequence, said expression being further detected and/or measured. A positive detection of the expression of said reporter DNA sequence means that an active transcription factor is formed, due to the binding together of said first influenza virus protein and second host cell protein.

Usually, in a two-hybrid assay, the first and second portion of a transcription factor consist respectively of (i) the DNA binding domain of a transcription factor and (ii) the activator domain of a transcription factor. In some embodiments, the DNA binding domain and the activator domain both originate from the same naturally occurring transcription factor. In some embodiments, the DNA binding domain and the activator domain originate from distinct naturally occurring factors, while, when bound together, these two portions form an active transcription factor. The term “portion” when used herein for transcription factor, encompasses complete proteins involved in multi protein transcription factors, as well as specific functional protein domains of a complete transcription factor protein.

Therefore in one embodiment of the invention, the assay of the invention comprises the following steps:

(1) providing a host cell expressing:
a first fusion polypeptide between (i) a ICN1 protein as defined above and (ii) a first protein portion of transcription factor
a second fusion polypeptide between (i) a nuclear protein as defined above and (ii) a second portion of a transcription factor

said transcription factor being active on DNA target regulatory sequence when the first and second protein portion are bound together and

said host cell also containing a nucleic acid comprising (i) a regulatory DNA sequence that may be activated by said active transcription factor and (ii) a DNA report sequence that is operatively linked to said regulatory sequence

(2) bringing said host cell provided at step 1) into contact with a test compound to be tested

(3) determining the expression level of said DNA reporter sequence.

The expression level of said DNA reporter sequence that is determined at step (3) above is compared with the expression of said DNA reporter sequence when step (2) is omitted. A different expression level of said DNA reporter sequence in the presence of the test compound means that the said test substance effectively inhibits the binding between the ICN1 protein and the nuclear protein and that said test compound may be positively selected.

Suitable host cells include, without limitation, prokaryotic cells (such as bacteria) and eukaryotic cells (such as yeast cells, mammalian cells, insect cells, plant cells, etc.). However preferred host cell are yeast cells and more preferably a Saccharomyces cerevisiae cell or a Schizosaccharomyces pombe cell.

Similar systems of two-hybrid assays are well known in the art and therefore can be used to perform the assay according to the invention (see. Fields et al. 1989; Vasavada et al. 1991; Fearon et al. 1992; Dang et al., 1991, Chien et al. 1991, U.S. Pat. No. 5,283,173, U.S. Pat. No. 5,667,973, U.S. Pat. No. 5,468,614, U.S. Pat. No. 5,525,490 and U.S. Pat. No. 5,637,463). For instance, as described in these documents, the Gal4 activator domain can be used for performing the assay according to the invention. Gal4 consists of two physically discrete modular domains, one acting as the DNA binding domain, the other one functioning as the transcription-activation domain. The yeast expression system described in the foregoing documents takes advantage of this property. The expression of a Gall-LacZ reporter gene under the control of a Gal4-activated promoter depends on the reconstitution of Gal4 activity via protein-protein interaction. Colonies containing interacting polypeptides are detected with a chromogenic substrate for β-galactosidase. A compete kit (MATCHMAKER™) for identifying protein-protein interactions is commercially available from Clontech.

So in one embodiment, the ICN1 protein as above defined is fused to the DNA binding domain of Gal4 and the nuclear protein as above defined is fused to the activation domain of Gal4.

The expression of said detectable marker gene may be assessed by quantifying the amount of the corresponding specific mRNA produced. However, usually the detectable marker gene sequence encodes for detectable protein, so that the expression level of the said detectable marker gene is assessed by quantifying the amount of the corresponding protein produced. Techniques for quantifying the amount of mRNA or protein are well known in the art. For example, the detectable marker gene placed under the control of regulatory sequence may consist of the β-galactosidase as above described.

### Western Blotting

In another one embodiment, the assay according to the invention comprises a step of subjecting to a gel migration assay the mixture of the ICN1 protein and the nuclear protein as above defined, with or without the test compound to be tested and then measuring the binding of said proteins altogether by performing a detection of the complexes formed between said proteins. The gel migration assay can be carried out as known by the one skilled in the art.

Therefore in one embodiment of the invention, the assay of the invention comprises the following steps:

(1) providing the ICN1 protein and the nuclear protein as defined above,

(2) bringing into contact the test compound to be tested with said proteins,

(3) performing a gel migration assay a suitable migration substrate with said proteins and said test compound as obtained at step (2), and

(4) detecting and quantifying the complexes formed between said proteins on the migration assay as performed at step (3).

The presence or the amount of the complexes formed between the proteins is then compared with the results obtained when the assay is performed in the absence of the test compound to be tested. Therefore, when no complexes between the proteins is detected or, alternatively when those complexes are present in a lower amount compared to the amount obtained in the absence of the test compound, means that the test compound may be selected as an inhibitor of the specific interaction between said ICN1 protein and said nuclear protein.

The detection of the complexes formed between the said two proteins may be easily performed by staining the migration gel with a suitable dye and then determining the protein bands corresponding to the protein analysed since the complexes formed between the first and the second proteins possess a specific apparent molecular weight. Staining of proteins in gels may be done using the standard Coomassie brilliant blue (or PAGE blue), Amido Black, or silver stain reagents of different kinds. Suitable gels are well known in the art such as sodium dodecyl (lauryl) sulfate-polyacrylamide gel. In a general manner, western blotting assays are well known in the art and have been widely described (Rybicki et al., 1982; Towbin et al. 1979; Kurien et al. 2006).

In a particular embodiment, the protein bands corresponding to the proteins submitted to the gel migration assay can be detected by specific antibodies. It may used both antibodies directed against the influenza virus proteins and antibodies specifically directed against the host cell proteins.

In another embodiment, the said two proteins are labelled with a detectable antigen as above described. Therefore, the proteins bands can be detected by specific antibodies directed against said detectable antigen. Preferably, the detectable antigen conjugates to the ICN1 protein is different from the antigen conjugated to the nuclear protein. For instance, the ICN1 protein can be fused to a GST detectable antigen and the nuclear protein can be fused with the HA antigen. Then the protein complexes formed between the two proteins may be quantified and determined with antibodies directed against the GST and HA antigens respectively.

### Biosensor Assays

In another embodiment, the assay of the present invention includes the use of an optical biosensor such as described by Edwards et al. (1997) or also by Szabo et al. (1995). This technique allows the detection of interactions between molecules in real time, without the need of labelled molecules. This technique is indeed bases on the surface plasmon resonance (SPR) phenomenon. Briefly, a first protein partner is attached to a surface (such as a carboxymethyl dextran matrix). Then the second protein partner is incubated with the previously immobilised first partner, in the presence or absence of the test substance to be tested. Then the binding including the binding level or the absence of binding between said protein partners is detected. For this purpose, a light beam is directed towards the side of the surface area of the substrate that does not contain the sample to be tested and is reflected by said surface. The SPR phenomenon causes a decrease in the intensity of the reflected light with a combination of angle and wavelength. The binding of the first and second protein partner causes a change in the refraction index on the substrate surface, which change is detected as a change in the SPR signal.

### Affinity Chromatography

In another one embodiment of the invention, the assay includes the use of affinity chromatography.

Test compounds for use in the assay above can also be selected by any immunoaffinity chromatography technique using any chromatographic substrate onto which (i) the ICN1 protein or (ii) the nuclear protein as above defined, has previously been immobilised, according to techniques well known from the one skilled in the art. Briefly, the ICN1 or the nuclear protein as above defined, may be attached to a column using conventional techniques including chemical coupling to a suitable column matrix such as agarose, Affi Gel®, or other matrices familiar to those of skill in the art. In some embodiment of this method, the affinity column contains chimeric proteins in which the ICN1 protein or nuclear protein as above defined, is fused to glutathion-s-transferase (GST). Then a test compound is brought into contact with the chromatographic substrate of the affinity column previously, simultaneously or subsequently to the other protein among the said first and second protein. The after washing, the chromatography substrate is eluted and the collected elution liquid is analysed by detection and/or quantification of the said later applied first or second protein, so as to determine if, and/or to which extent, the test substance has impaired or not the binding between (i) the ICN1 protein and (ii) the nuclear protein.

In another one embodiment of the assay according to the invention, the ICN1 protein and the nuclear protein as above defined are labelled with a fluorescent molecule or substrate. Therefore, the potential alteration effect of the test compound to be tested on the binding between ICN1 protein and the nuclear protein as above defined is determined by fluorescence quantification.

For example, the ICN1 protein and the nuclear protein as above defined may be fused with auto-fluorescent polypeptides, as GFP or YFPs as above described. The first ICN1 protein and the nuclear protein as above defined may also be labelled with fluorescent molecules that are suitable for performing fluorescence detection and/or quantification for the binding between said proteins using fluorescence energy transfer (FRET) assay. The ICN1 protein and the nuclear protein as above defined may be directly labelled with fluorescent molecules, by covalent chemical linkage with the fluorescent molecule as GFP or YFP. The ICN1 protein and the nuclear protein as above defined may also be indirectly labelled with fluorescent molecules, for example, by non covalent linkage between said polypeptides and said fluorescent molecule. Actually, said ICN1 protein and nuclear protein as above defined may be fused with a receptor or ligand and said fluorescent molecule may be fused with the corresponding ligand or receptor, so that the fluorescent molecule can non-covalently bind to said first influenza virus protein and second host cell protein. A suitable receptor/ligand couple may be the biotin/streptavifin paired member or may be selected among an antigen/antibody paired member. For example, a protein according to the invention may be fused to a poly-histidine tail and the fluorescent molecule may be fused with an antibody directed against the poly-histidine tail.

### Fluorescence Assays

As already specified, the assay according to the invention encompasses determination of the ability of the test compound to inhibit the interaction between the ICN1 protein and the nuclear protein as above defined by fluorescence assays using FRET. Thus, in a particular embodiment, the ICN1 protein as above defined is labelled with a first fluorophore substance and the nuclear protein is labelled with a second fluorophore substance. The first fluorophore substance may have a wavelength value that is substantially equal to the excitation wavelength value of the second fluorophore, whereby the bind of said first and second proteins is detected by measuring the fluorescence signal intensity emitted at the emission wavelength of the second fluorophore substance. Alternatively, the second fluorophore substance may also have an emission wavelength value of the first fluorophore, whereby the binding of said ICN1 protein and said nuclear protein is detected by measuring the fluorescence signal intensity emitted at the wavelength of the first fluorophore substance.

The fluorophores used may be of various suitable kinds, such as the well-known lanthanide chelates. These chelates have been described as having chemical stability, long-lived fluorescence (greater than 0.1 ms lifetime) after bioconjugation and significant energy-transfer in specificity bioaffinity assay. Document U.S. Pat. No. 5,162,508 discloses bipyridine cryptates. Polycarboxylate chelators with TEKES type photosensitizers (EP0203047A1) and terpyridine type photosensitizers (EP0649020A1) are known. Document WO96/00901 discloses diethylenetriaminepentaacetic acid (DPTA) chelates which used carbostyril as sensitizer. Additional DPT chelates with other sensitizer and other tracer metal are known for diagnostic or imaging uses (e.g., EPO450742A1).

In a preferred embodiment, the fluorescence assay consists of a Homogeneous Time Resolved Fluorescence (HTRF) assay, such as described in document WO 00/01663 or U.S. Pat. No. 6,740,756, the entire content of both documents being herein incorporated by reference. HTRF is a TR-FRET based technology that uses the principles of both TRF (time-resolved fluorescence) and FRET. More specifically, the one skilled in the art may use a HTRF assay based on the time-resolved amplified cryptate emission (TRACE) technology as described in Leblanc et al. (2002). The HTRF donor fluorophore is Europium Cryptate, which has the long-lived emissions of lanthanides coupled with the stability of cryptate encapsulation. XL665, a modified allophycocyanin purified from red algae, is the HTRF primary acceptor fluorophore. When these two fluorophores are brought together by a biomolecular interaction, a portion of the energy captured by the Cryptate during excitation is released through fluorescence emission at 620 nm, while the remaining energy is transferred to XL665. This energy is then released by XL665 as specific fluorescence at 665 nm. Light at 665 nm is emitted only through FRET with Europium. Because Europium Cryptate is always present in the assay, light at 620 nm is detected even when the biomolecular interaction does not bring XL665 within close proximity.

Therefore, in one embodiment, the assay may therefore comprises the steps of:

(1) bringing into contact a pre-assay sample comprising:
the ICN1 protein fused to a first antigen,
a nuclear protein fused to a second antigen,
a test compound to be tested

(2) adding to the said pre assay sample of step (1):
at least one antibody labelled with a European Cryptate which is specifically directed against the first said antigen,
at least one antibody labelled with XL665 directed against the second said antigen,

(3) illuminating the assay sample of step (2) at the excitation wavelength of the said European Cryptate,

(4) detecting and/or quantifying the fluorescence signal emitted at the XL665 emission wavelength, and

(5) comparing the fluorescence signal obtained at step (4) to the fluorescence obtained wherein pre assay sample of step (1) is prepared in the absence of the test compound to be tested.

If at step (5) as above described, the intensity value of the fluorescence signal is lower than the intensity value of the fluorescence signal found when pre assay sample of step (1) is prepared in the absence of the test compound to be tested, then the test substance may be selected as an inhibitor of the specific interaction between said ICN1 protein and said nuclear protein.

Antibodies labelled with a European Cryptate or labelled with XL665 can be directed against different antigens of interest including GST, poly-histidine tail, DNP, c-myx, HA antigen and FLAG which include. Such antibodies encompass those which are commercially available from CisBio (Bedfors, Mass., USA), and notably those referred to as 61GSTKLA or 61HISKLB respectively.

Alternatively, in another one embodiment of the assay according to the invention, the modulation of the specific interaction between the ICN1 protein and the nuclear protein may be determined using isothermal titration calorimetry (ITC). Typically, ITC experiments were performed with an ITC titration calorimeter (such as provide by Microcal Inc., Northampton, Mass., USA). Solutions comprising the ICN1 protein (or alternatively the nuclear protein) are then prepared. The enthalpy change resulting from the contacting with the nuclear protein (or alternatively the ICN1 protein) was obtained through integration of the calorimetric signal. Different ITC experimental formats are typically employed in order to obtain compound dissociation constants (Kd's) over a wide range of affinities.

Assays for Identifying a Compound that Inhibits the Expression of a Nuclear Protein as Depicted in Table 1:

Another particular aspect of the invention relates to an assay for identifying a compound that inhibits the expression of a host cell protein as depicted in Table 1 comprising determining whether the test compound inhibits the expression of said nuclear protein

In one embodiment, the assay comprises the steps of i) contacting the test compound with a cell transfected with a reporter gene operatively linked to all or part of the promoter of the gene encoding for the nuclear protein, ii) assessing the level of expression of said reporter gene, and iii) identifying the test compound which inhibits the expression of said reporter gene.

Abroad variety of host-expression vector systems may be utilized to express the genes used in the assay. These include, but are not limited to, mammalian cell systems such as human cell lines derived from colon adenocarcinoma including HT-29, Caco-2, SW480, HTC116, The mammalian cell systems may harbour recombinant expression constructs containing promoters derived from the genome of mammalian cells or from mammalian viruses (e.g., the adenovirus late promoter or the vaccine virus 7.5K promoter).

Additional host-expression vector systems include, but are not limited to, microorganisms such as bacteria (e.g., E. coli or B. subtilis) transformed with recombinant bacteriophage DNA, plasmid DNA, or cosmid DNA expression vectors containing PTK or adaptor protein coding sequences; yeast (e.g., Saccharomyces, Pichia) transformed with recombinant yeast expression vectors containing the protein or peptide oding sequences; insect cell systems, such as Sf9 or Sf21 infected with recombinant virus expression vectors (e.g., baculovirus) containing the protein or peptide coding sequences; amphibian cells, such as Xenopus oocytes; or plant cell systems infected with recombinant virus express-15 sion vectors (e.g., cauliflower mosaic virus, CaMV; tobacco mosaic virus, TMV) or transformed with recombinant plasmid expression vectors (e.g., Ti plasmid) containing the protein or peptide coding sequence. Culture conditions for each of these cell types is specific and is known to those familiar with the art.

DNA encoding proteins to be assayed can be transiently or stably expressed in the cell lines by several methods known in the art, such as, calcium phosphate-mediated, DEAE-dextran mediated, liposomal-mediated, viral-mediated, electroporation-mediated and microinjection delivery. Each of these methods may require optimization of assorted experimental parameters depending on the DNA, cell line, and the type of assay to be subsequently employed.

In addition native cell lines that naturally carry and express the nucleic acid sequences for the target protein may be used.

In a particular embodiment, the invention is directed to a method, which comprises the steps of i) contacting the test compound with a cell capable of expressing the gene encoding for the nuclear protein, ii) assessing the level of expression of said gene, and iii) identifying the test compound which inhibits the expression of said gene. In one embodiment, the level of expression is assessed by determining the level of transcription of said gene. In a further embodiment, the determination of the level of translation of said gene is effected by means of an immunoassay.

Determination of the expression level of a gene can be performed by a variety of techniques. Generally, the expression level as determined is a relative expression level.

More preferably, the determination comprises contacting the sample with selective reagents such as probes, primers or ligands, and thereby detecting the presence, or measuring the amount, of polypeptide or nucleic acids of interest originally in the sample. Contacting may be performed in any suitable device, such as a plate, microtiter dish, test tube, well, glass, column, and so forth In specific embodiments, the contacting is performed on a substrate coated with the reagent, such as a nucleic acid array or a specific ligand array. The substrate may be a solid or semi-solid substrate such as any suitable support comprising glass, plastic, nylon, paper, metal, polymers and the like. The substrate may be of various forms and sizes, such as a slide, a membrane, a bead, a column, a gel, etc. The contacting may be made under any condition suitable for a detectable complex, such as a nucleic acid hybrid or an antibody-antigen complex, to be formed between the reagent and the nucleic acids or polypeptides of the sample.

In a preferred embodiment, the expression level may be determined by determining the quantity of mRNA.

Methods for determining the quantity of mRNA are well known in the art. For example the nucleic acid contained in the samples (e.g., cell or tissue prepared from the subject) is first extracted according to standard methods, for example using lytic enzymes or chemical solutions or extracted by nucleic-acid-binding resins following the manufacturer's instructions. The extracted mRNA is then detected by hybridization (e.g., Northern blot analysis) and/or amplification (e.g., RT-PCR). Preferably quantitative or semi-quantitative RT-PCR is preferred. Real-time quantitative or semi-quantitative RT-PCR is particularly advantageous.

Other methods of Amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization. A wide variety of appropriate indicators are known in the art including, fluorescent, radioactive, enzymatic or other ligands (e.g. avidin/biotin).

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In another preferred embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample comprising cells as above defined above, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210)

Other methods for determining the expression level of said genes include the determination of the quantity of proteins encoded by said genes.

Such methods comprise contacting a biological sample with a binding partner capable of selectively interacting with a marker protein present in the sample. The binding partner is generally an antibody that may be polyclonal or monoclonal, preferably monoclonal.

The presence of the protein can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith.

Such immunoassays generally involve separation of unbound protein in a liquid phase from a solid phase support to which antigen-antibody complexes are bound. Solid supports which can be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like.

More particularly, an ELISA method can be used, wherein the wells of a microtiter plate are coated with an antibody against the protein to be tested. A biological sample containing or suspected of containing the marker protein is then added to the coated wells. After a period of incubation sufficient to allow the formation of antibody-antigen complexes, the plate (s) can be washed to remove unbound moieties and a detectably labeled secondary binding molecule added. The secondary binding molecule is allowed to react with any captured sample marker protein, the plate washed and the presence of the secondary binding molecule detected using methods well known in the art.

Assays for Identifying a Compound that Inhibits the Activity of a Nuclear Protein as Depicted in Table 1:

Another particular aspect of the invention relates to an assay for identifying a compound that inhibits the activity of a nuclear protein as depicted in Table 1 comprising:

(a) contacting the nuclear protein with a test compound, and

(b) determining whether the test compound inhibits the activity of said nuclear protein.

The activity of a nuclear protein may be easily determined by the skilled man in the art. For example, for enzymes, various enzymatic assay may be used for determined whether the test compound could inhibits the activity of said nuclear protein. Other functional assays may be used and may be determined by the information disclosed in the prior art.

In one particular embodiment, the nuclear protein required for Notch1 transcriptional activity is selected from the group consisting of PHF8 and AF4p12.

Test Compounds of the Invention:

According to one embodiment of the invention, the test compound of may be selected from the group consisting of peptides, peptidomimetics, small organic molecules, antibodies, aptamers or nucleic acids. For example, the test compound according to the invention may be selected from a library of compounds previously synthesised, or a library of compounds for which the structure is determined in a database, or from a library of compounds that have been synthesised de novo.

In a particular embodiment, the test compound may be selected form small organic molecules.

As used herein, the term “small organic molecule” refers to a molecule of size comparable to those organic molecules generally sued in pharmaceuticals. The term excludes biological macromolecules (e.g.; proteins, nucleic acids, etc.); preferred small organic molecules range in size up to 2000 Da, and most preferably up to about 1000 Da.

In another particular embodiment, the test compound according to the invention may be antibodies specifically directed to the interaction site between ICN1 and a nuclear protein required for Notch1 transcriptional activity as depicted in Table 1 or impacting their interaction and/or their cellular functions (e.g. the transcription of Notch-target genes).

The term “antibody” or “antibodies” relates to an antibody characterized as being specifically directed to the interaction site of ICN1 with a nuclear protein required for Notch1 transcriptional activity of the invention, or any functional derivative thereof, with above mentioned antibodies being preferably monoclonal antibodies; or an antigen-binding fragment thereof, of the F (ab′)2, or single chain Fv type, or any type of recombinant antibody derived thereof. These antibodies of the invention include specific polyclonal antisera prepared against the interaction site of ICN1 with a nuclear protein of the invention.

The antibodies of the invention can for instance be produced by any hybridoma liable to be formed according to classical methods from splenic cells of an animal, particularly of a mouse or rat immunized against the peptidic sequence involved in the interaction between ICN1 and a nuclear protein of the invention or any functional derivative thereof, and of cells of a myeloma cell line, and to be selected by the ability of the hybridoma to produce the monoclonal antibodies recognizing the peptidic sequence involved in the interaction between ICN1 and a nuclear protein of the invention or any functional derivative thereof which have been initially used for the immunization of the animals. The antibodies according to this embodiment of the invention may be humanized versions of the mouse antibodies made by means of recombinant DNA technology, departing from the mouse and/or human genomic DNA sequences coding for H and L chains or from cDNA clones coding for H and L chains.

Alternatively, the antibodies according to this embodiment of the invention may be human antibodies. Such human antibodies are prepared, for instance, by means of human peripheral blood lymphocytes (PBL) repopulation of severe combined immune deficiency (SCID) mice as described in PCT/EP99/03605 or by using transgenic non-human animals capable of producing human antibodies as described in U.S. Pat. No. 5,545,806. Also fragments derived from these antibodies such as Fab, F (ab)′2 ands (“single chain variable fragment”), providing they have retained the original binding properties, form part of the present invention. Such fragments are commonly generated by, for instance, enzymatic digestion of the antibodies with papain, pepsin, or other proteases. It is well known to the person skilled in the art that monoclonal antibodies or fragments thereof, can be modified for various uses. An appropriate label of the enzymatic, fluorescent, or radioactive type can label the antibodies involved in the invention.

In another particular embodiment, the test compound according to the invention may be selected from aptamers. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996).

In still another particular embodiment, the test compound may be selected from molecules that block the synthesis of a nuclear protein required for Notch1 transcriptional activity as depicted in Table 1.

By synthesis is meant the transcription of the gene of interest coding for a nuclear protein required for Notch1 transcriptional activity as depicted in Table 1. Small molecules can bind on the promoter region of the gene of interest and inhibit binding of a transcription factor or these molecules can bind a transcription factor and inhibit binding to the gene-promoter so that there is no expression of the gene of interest.

Also within the scope of the invention is the use of oligoribonucleotide sequences that include anti-sense RNA and DNA molecules and ribozymes that function to inhibit the translation of mRNA of a nuclear protein required for Notch1 transcriptional activity. Anti-sense RNA and DNA molecules act to directly block the translation of mRNA by binding to targeted mRNA and preventing protein translation. In regard to antisense DNA, oligodeoxyribonucleotides derived from the translation initiation site. Ribozymes are enzymatic RNA molecules capable of catalysing the specific cleavage of RNA. The mechanism of ribozyme action involves sequence specific hybridisation of the ribozyme molecule to complementary target RNA, followed by an endonucleolytic cleavage.

To inhibit the activity of the gene of interest or the gene product of the gene of interest, custom-made techniques are available directed at three distinct types of targets: DNA, RNA and protein. For example, the gene or gene product of a nuclear protein required for Notch1 transcriptional activity of the invention can be altered by homologous recombination, the expression of the genetic code can be inhibited at the RNA levels by antisense oligonucleotides, interfering RNA (RNAi) or ribozymes, and the protein function can be altered by antibodies or drugs.

Methods for Screening a Compound Capable of Inhibiting the Notch1 Transcriptional Activity:

The test compounds that have been positively selected at the end of any one of the embodiments of the in vitro screening which has been described previously in the present specification may be subjected to further selection steps in view of further assaying their properties on the Notch1 transcriptional activity (e.g. inhibition of the transcription of Notch-target genes such as HES1, NOTCH3, CR2, IL7R, DTX1, and HEY1).

A particular aspect of the present invention thus relates to method for screening a compound useful for inhibiting the Notch1 transcriptional activity comprising the steps consisting of (a) selecting a test compound by performing at least one assay as described above (b) determining whether said compound inhibits the Notch1 transcriptional activity in a cell and (c) positively selecting the test compound capable of inhibiting the Notch1 transcriptional activity in a cell.

In one embodiment, the method comprises the steps consisting of i) culturing a cell in presence of the test compound, ii) comparing the Notch1 transcriptional activity in said cell with the Notch1 transcriptional activity determined in the absence of the test compound and iii) positively selecting the test compound that provides a decrease in the Notch1 transcriptional activity.

The term “decrease in the Notch1 transcriptional activity” as used herein with reference to the transcription of Notch-target genes in a cell, means that the transcription of Notch-target genes in a cell is lower in the presence of a compound as above described relative to the transcription of Notch-target genes in a cell in the absence of said substance. In one embodiment, the presence of said compound which will inhibit transcription of Notch-target genes by at least about 10%, or by at least about 20%, or by at least about 30%, or by at least about 40%, or by at least about 50%, or by at least about 60%, or by at least about 70%, or by at least about 80%, or by at least about 90%, or by at least about 100%, or by at least about 200%, or by at least about 300%, or by at least about 400%, or by at least about 500% when compared to the transcription of Notch-target genes in the absence of said compound. Said Notch1 transcriptional activity may be typically determined by assessing the level of expression of Notch-target genes. Non-limiting examples of Notch-target genes that can be suitable for the invention include but are not limited to HES1, NOTCH3, CR2, IL7R, DTX1, ID1, RCBTB2 and HEY1. Determination of the expression level of a gene can be performed by a variety of techniques as previously described.

### Cells Used in the Screening Methods

According to the invention, any eukaryotic cell may be used in the screening method of the invention. Preferably said cell is a mammalian cell. Typically said mammalian cells include but are not limited to cells from humans, dogs, cats, cattle, horses, sheep, pigs, goats, and rabbits. In a particular embodiment the cell is a human cell. In another particular embodiment said cell is a cell line. Non-limiting examples of cell lines that can be suitable for the invention include but are not limited to T-cell acute lymphoblastic leukemia (T-ALL) cell lines (such as SupT1, HPB-ALL, TALL1, DND41, MOLT4 and H9).

Typically, cells are cultured in a standard commercial culture medium, such as Dulbecco's modified Eagle's medium supplemented with serum (e.g., 10% fetal bovine serum), or in serum free medium, under controlled humidity and C02 concentration suitable for maintaining neutral buffered pH (e.g., at pH between 7.0 and 7.2). Suitable serum free media are described, for example, in U.S. Provisional Application No. 60/638,166, filed Dec. 23, 2004. Optionally, the medium contains antibiotics to prevent bacterial growth, e.g., penicillin, streptomycin, etc., and/or additional nutrients, such as L-glutamine, sodium pyruvate, nonessential amino acids, additional supplements to promote favorable growth characteristics, e.g., trypsin, 3-mercaptoethanol, and the like.

### Methods of Prevention or Treatment of the Invention

As described above, the methods of the present invention are particularly useful for screening a compound that may be used for the treatment or prevention of cell proliferative diseases and disorders, including certain forms of cancer, associated with overexpression and/or activation of Notch1 as described infra.

In a further aspect, the present invention thus provides a method for the prevention or treatment of a cell proliferative disease and disorder associated with overexpression and/or activation of Notch1 comprising administering to a patient in need thereof a therapeutically effective amount of a compound that inhibits the interaction between the ICN1 proteins and a nuclear protein required for Notch1 transcriptional activity as depicted in Table 1. Said compound may be identified by the screening methods of the invention.

More particularly, the present invention relates to a compound that inhibits the interaction between ICN1 and a nuclear protein required for Notch1 transcriptional activity as depicted in Table 1 for use in the prevention or treatment of a cell proliferative disease and disorder associated with overexpression and/or activation of Notch1.

In one particular embodiment, the compound that inhibits the interaction between ICN1 and a nuclear protein required for Notch1 transcriptional activity as depicted in Table 1 is an inhibitor of gene expression.

Inhibitors of gene expression for use in the present invention may be based on anti-sense oligonucleotide constructs. Anti-sense oligonucleotides, including anti-sense RNA molecules and anti-sense DNA molecules, would act to directly block the translation of mRNA by binding thereto and thus preventing protein translation or increasing mRNA degradation, thus decreasing the level of a nuclear protein of the invention, and thus activity, in a cell. For example, antisense oligonucleotides of at least about 15 bases and complementary to unique regions of the mRNA transcript sequence encoding a nuclear protein of the invention can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of gene expression of a nuclear protein of the invention for use in the present invention. Gene expression can be reduced by contacting a subject or cell with a small double stranded RNA (dsRNA), or a vector or construct causing the production of a small double stranded RNA, such that gene expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

In one embodiment, the nuclear protein required for Notch1 transcriptional activity is selected from the group consisting of PHF8, AF4p12, LSD1 and BRG1.

Accordingly, the present invention relates to an inhibitor of PHF8, AF4p12, LSD1 or BRG1 gene expression for use in the prevention or treatment of a cell proliferative disease and disorder associated with overexpression and/or activation of Notch1.

The present invention also related to a method for the prevention or treatment of a cell proliferative disease and disorder associated with overexpression and/or activation of Notch1 comprising administering to a patient in need thereof a therapeutically effective amount of an inhibitor of PHF8, AF4p12, LSD1 or BRG1 gene expression.

In one particular embodiment, the sequence of the shRNA targeting PHF8 is represented by SEQ ID NO: 1.

In another particular embodiment, the sequence of the shRNA targeting AF4p12 is represented by SEQ ID NO: 2.

In another particular embodiment, the sequence of the shRNA targeting AF4p12 is represented by SEQ ID NO: 3.

In another particular embodiment, the sequence of the shRNA targeting LSD1 is represented by SEQ ID NO: 2.

In another particular embodiment, the sequence of the shRNA targeting LSD1 is represented by SEQ ID NO: 4.

In still another particular embodiment, the sequence of the shRNA targeting BRG1 is represented by SEQ ID NO: 5.

In still another particular embodiment, the sequence of the shRNA targeting BRG1 is represented by SEQ ID NO: 6.

Ribozymes can also function as inhibitors gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of mRNA sequences of a nuclear protein of the invention are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of gene expression of a nuclear protein of the invention can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing a nuclear protein of the invention. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles are provided in Kriegler, 1990 and in Murry, 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g. Sambrook et al., (1989). In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In still a further aspect, the present invention thus provides a method for the prevention or treatment of a cell proliferative disease and disorder associated with overexpression and/or activation of Notch1 comprising administering to a patient in need thereof a therapeutically effective amount of a compound that inhibits the activity (such as enzymatic activity) of a nuclear protein required for Notch1 transcriptional activity as depicted in Table 1.

Said compound may be identified by the screening methods of the invention.

In one embodiment, the nuclear protein required for Notch1 transcriptional activity is selected from the group consisting of PHF8 and AF4p12.

Accordingly, the present invention relates to an inhibitor of PHF8 or AF4p12 activity for use in the prevention or treatment of a cell proliferative disease and disorder associated with overexpression and/or activation of Notch1.

The compounds of the invention are useful for the prevention and treatment of a cell proliferative disease and disorder associated with overexpression and/or activation of Notch1 including breast cancer, ovarian cancer, prostate cancer, cervical cancer, lung cancer, brain cancers (e.g., glioblastoma, astrocytoma, neuroblastoma), melanomas, gastrointestinal cancers (e.g., colorectal, pancreatic, and gastric), head and neck cancer, and hematopoietic cell cancers, (e.g., multiple myeloma, leukemia, e.g., T-cell acute lymphoblastic leukemia (T-ALL), precursor B acute lymphoblastic leukemia (B-ALL) and B-cell chronic lymphoblastic leukemia (B-CLL)).

In one embodiment, the cell proliferative disease and disorder associated with overexpression and/or activation of Notch1 is T-ALL.

According to the invention, the term “patient in need thereof” is intended for a human or non-human mammal (e.g. dog, cat, horses . . . ) affected or likely to be affected with ell proliferative disease and disorder associated with overexpression and/or activation of Notch1

By a “therapeutically effective amount” of the compound of the invention is meant a sufficient amount of compound to treat cell proliferative diseases and disorders associated with overexpression and/or activation of Notch1, at a reasonable benefit/risk ratio applicable to any medical treatment. It will be understood, however, that the total daily usage of the compound of the invention and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than those required to achieve the desired therapeutic effect and to gradually increase the dosage until the desired effect is achieved.

The compound that inhibits the interaction between ICN1 and a nuclear protein required for Notch1 transcriptional activity of the invention may be combined with pharmaceutically acceptable excipients. “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The invention will be further illustrated by the following figures and examples. However, these examples and figures should not be interpreted in any way as limiting the scope of the present invention.

### FIGURES

FIG. 1: Identification of Notch1-associated nuclear factors: Interaction network of ICN1-associated proteins identified by Mass Spectrometry (MS). The false positive interactors were excluded by removing all proteins that were also detected in the control purification. See also Table 1.

FIG. 2: PBAF, LSD1, PHF8 and AF4p12 associate with ICN1-CSL-MAML1: (A) Flag and HA-immunopurified ICN1-associated proteins from SupT1 nuclear extracts (NEs) were resolved on SDS-PAGE and the presence of partners identified by mass spectrometry was confirmed by western blot (WB). (B) NEs from SupT1 stably expressing LSD1-F (Flag-tagged), PHF8-F or BRG1-F were subjected to immunoprecipitation (IP) using anti-Flag beads. The presence of endogenous ICN1 and CSL in the purified material was revealed by WB. The anti-ICN1 antibody specifically recognizes the γ-secretase-cleaved active form of NOTCH1 (V1744). (C) Interaction between endogenous LSD1, PHF8 or BRG1 with components of the Notch-activation complex. LSD1, PHF8 and BRG1 were purified from SupT1 NEs using specific antibodies and the presence of ICN1 or CSL in the purified material was revealed by WB. (D) SupT1 cells stably expressing Flag and HA-tagged MAML1 (MAML1-F/H) were treated with DMSO or GSI (500 nM, 8 hours). MAML1-associated proteins were Flag-HA immunopurified from NEs and analyzed by WB using the indicated antibodies. (E) BRG1, PB1, LSD1, PHF8 and AF4p12 are associated with the Notch-activation complex. NEs from SupT1 stably transduced with Flag-MAML1 and HA-ICN1 were subjected to sequential IP using anti-Flag and anti-HA beads. The presence of Notch cofactors in the purified material was analyzed by WB. (F) Notch cofactors assemble into a single complex containing ICN1-CSL-MAML1. Reciprocal IPs with anti-Flag and anti-HA beads were performed using NEs from SupT1 cells stably coexpressing HA-ICN1 and Flag-PHF8, -LSD1 or -BRG1. Eluates were subjected to WB.

FIG. 3: Notch-associated cofactors are required for Notch transcriptional responses: BRG1, LSD1, PHF8 and AF4p12 regulate Notch-mediated activation of its target genes in T-ALL. SupT1 cells expressing specific shRNAs were further treated with DMSO or GSI (1 μM, 24 hrs). Notch-induced expression of eight direct target genes was measured by quantitative RT-PCR. mRNA levels were normalized to GAPDH mRNAs and represented relative to their expression level in the absence of Notch (GSI-treated cells). Shown are means+/−SD (n≧3).

FIG. 4: AF4p12 is a Notch transcriptional coactivator: (A) AF4p12 is required for Notch-target genes expression in T-ALL cell lines. TALL1, HPB-ALL and DND41 were transduced with control, CSL or AF4p12 shRNA. Expression of HES1 and IL7R was measured by quantitative (Q-)RT-PCR (mean+/−SD, n=2). (B) AF4p12 affects the rate of transcription of Notch-target genes. Nuclear run-on assays (n=3) were performed on isolated nuclei from SupT1 cells expressing control or AF4p12 shRNAs. Transcripts generated during the run-on were purified using anti-BrdU beads and analyzed by Q-RT-PCR. (C) AF4p12 controls Notch transcriptional activity in transient reporter assay. HeLa cells expressing control or AF4p12 shRNA were transfected with a Notch-responsive luciferase reporter (p6XCBS-luc) and various amounts of ICN1 expression vector. The values are Relative Luciferase Units (RLU) represented as fold induction by ICN1 (mean+/−SD, n=2).

FIG. 5: Opposing role of LSD1 in the regulation of Notch-target genes: (A) LSD1 is required for ligand-mediated HES1 activation. U937 cells expressing control or LSD1 shRNA were cultured on pre-coated DL-4 Notch ligand (5 μg/mL) for 1 hr. HES1 expression was analyzed by Q-RT-PCR (n=3). WB analyses are shown in FIG. S4G. (B) LSD1 controls Notch-target genes expression in NOTCH1-dependent T-ALL cell lines. SupT1 and HPB-ALL were transduced with control or LSD1 shRNA. Expression of HES1, NOTCH3 and CR2 was measured by Q-RT-PCR (n=3). (C) Model for LSD1 functions in Notch-target genes regulation. All Q-RT-PCR were normalized to GAPDH mRNAs and are represented as mean+/−SD.

FIG. 6: PHF8 demethylase activity is required for Notch-mediated activation of its target genes: (A) PHF8 is required for Notch-responsive genes expression in T-ALL cell lines. HPB-ALL, TALL1, MOLT4, SupT1 and DND41 were transduced with control or PHF8 shRNA. Expression of DTX1 was measured by quantitative RT-PCR (Q-RT-PCR) and normalized to GAPDH. (B) mRNAs levels of IL7R, DTX1, HEY1 and CR2 were measured by Q-RT-PCR in SupT1 cells coexpressing PHF8 shRNA and the indicated PHF8 construct. Shown are means+/SD (n=3).

FIG. 7: Functional relevance of Notch cofactors in T-ALL proliferation and gene expression during T-cell development: (A) LSD1 and PHF8 are required for NOTCH1-dependent T-ALL cells growth. SupT1, HPB-ALL, TALL1 and DND41 cells were transduced with control, CSL, PHF8 or LSD1 specific shRNA. One week post-transduction, cell count proliferation assays were performed (n=3). The observed effects of CSL, PHF8 and LSD1 depletion is significant (p<0.05). (B) Notch-mediated expression of c-MYC in TALL cells requires PHF8 and LSD1. Quantitative RT-PCR analysis of c-MYC expression was performed in TALL1 expressing control, PHF8 or LSD1 shRNAs and treated with DMSO or GSI (n=3). (C) Depletion of PHF8 and LSD1 impairs T-ALL progression in vivo. SupT1 expressing control, CSL, LSD1 or PHF8 shRNA were implanted subcutaneously in SCID mice (n=5). Xenograft tumor volume was monitored over 21 days.

### EXAMPLE

### Material & Methods

Cell Culture and Treatment:

Human T-ALL cell lines SupT1, HPB-ALL, TALL1, DND41, MOLT4 and H9 were used in this study. NOTCH1 signaling in SupT1, HPB-ALL, TALL1, DND41 and MOLT4 is constitutively active and requires γ-secretase cleavage for activation. Notch signaling was inhibited by treating cells with the γ-secretase inhibitor (GSI) compound E (santa cruz) at a final concentration of 0.5-1 μM. For ligand-mediated Notch signaling activation, the monocytic cell line U937 was cultured for 1 hour with precoated recombinant Notch ligand Delta-like 4 (5 μg/mL). LSD1 demethylase activity was inhibited by addition of cell-permeable LSD1 inhibitors: tranylcypromine (TCP) and compound S2101. The general monoamine oxidase inhibitor TCP (Sigma P8511) was used at a final concentration of 1 mM. The recently designed compound S2101 (LSD1 Inhibitor II, Calbiochem), which exhibits stronger LSD1 inhibition (IC50=0.99 μM vs. 184 μM) and much weaker effect on monoamine oxidases (Mimasu et al., 2010), was used at 30 μM.

Expression Vectors:

Retroviral pOZ constructs containing a single tag (FLAG or HA) were made by modifying the pOZ-Flag/HA (F/H) vector (Nakatani and Ogryzko, 2003) and the pOZ.puro-F/H vector (Kumar et al., 2009). Human NOTCH1 intracellular domain (ICN1) was PCR amplified from the MigRI-ICN1 vector and inserted into the XhoI/NotI sites of pOZ vectors. pOZ-MAML1 and pOZ-LSD1 constructs were generated by PCR amplification of human MAML1 and LSD1 coding region from pFLAG-CMV2-MAML1 vector and pcDNA3-LSD1 vector pOZ-F/H vectors encoding human wide-type PHF8 and the catalytic mutant F278S were obtained from H. Qi and Y. Shi (Qi et al., 2010). These constructs contain silent mutations that confer shRNA resistance (R). PHF8 was subcloned into pOZ.puro vectors. pBABE-BRG1-Flag vector was obtained from Addgene (1959, Robert Kingston). All constructs were verified by sequencing.

Virus Production and Cell Line Transduction:

293T cells were transfected with a packaging mixture and the retroviral vector (pOZ, pSUPER, pBABE) using the calcium phosphate precipitation method. For transfection, 5 μg of the retroviral vector, 2.5 μg of the packaging plasmid (gag/pol) and 2.5 μg of the envelope plasmid were mixed with 100 μg of CaCl2 (1.25M) and 500 μL of HBS2X (sigma) in a final volume of 1 mL. The mixture was incubated 1 min at room temperature then added dropwise to the cells. The medium was changed the following day and the viral-containing supernatant was collected 48 hours after transfection, filtered through a 0.45 μm filter and subsequently used to infect cells.

To establish stable SupT1 cell lines expressing tagged ICN1, MAML1, LSD1, PHF8 or BRG1, we transduced SupT1 with recombinant retroviruses expressing a bicistronic mRNA that encodes the tagged protein and a selection marker (either IL-2 receptor subunit alpha or puromycin resistance gene). Transduced cells were purified by affinity cell sorting (for IL2R) or selected by puromycin treatment (2 μg/mL).

For shRNA-mediated knockdown experiments, cells were transduced with pSUPER retroviral vectors. After an overnight incubation, a second round of infection was performed using the same vector (for PHF8 and control shRNAs) or a second shRNA targeting the same mRNA (for CSL, LSD1, AF4p12 and BRG1). The medium was refreshed the following day and puromycin was added 72 hours post-infection at a final concentration of 2 μg/mL. Protein expression was analyzed by western blot after 3 days of selection. All the experiments were performed between day 6 and day 14 post-transduction.

Purification of Proteins Complexes:

Nuclear extracts were prepared using the Dignam protocol with slight modifications. For the purification of ICN1-associated complexes, 12×10⁹ SupT1 cells stably expressing Flag-HA tagged ICN1 and control SupT1 were harvested by centrifugation, washed in cold PBS and resuspended in 4 packed cell pellet volumes of hypotonic buffer (20 mM Tris-HCl pH 7.4, 10 mM NaCl, and 1.5 mM MgCl2). The suspension was incubated on ice for 10 min and then cells were lysed by 12 strokes using a Dounce homogenizer fitted with a B pestle. The nuclei were pelleted by centrifugation and resuspended in one packed nuclear pellet volume of a buffer containing 20 mM Tris-HCl pH 7.4, 300 mM NaCl, 25% glycerol, 0.2 mM EDTA, 1.5 mM MgCl2 and PMSF. One packed nuclear pellet volume of a high salt buffer (containing 20 mM Tris-HCl pH 7.4, 720 mM NaCl, 25% glycerol, 0.2 mM EDTA, 1.5 mM MgCl2 and PMSF) was added dropwise to the suspension gently stirring with a magnetic bar. After stirring for 30 min to allow extraction of transcription factors, the suspension was centrifuged at 13.000 g for 30 min at 4° C. and the supernatant was dialyzed against 100 volumes of buffer BC100 (20 mM Tris-HCl pH 7.4, 100 mM NaCl, 10% glycerol, 0.2 mM EDTA, 1.5 mM MgCl2 and PMSF) for 6 hours. The dialysate (nuclear extract) was cleared by centrifugation at 13.000 g for 30 min. Nuclear extracts were incubated for 4 hr (at 4° C. with rotation) with anti-FLAG M2 agarose beads (Sigma) (1% v/v) equilibrated in BC100. Beads were washed 3 times with 10 mL buffer B015 (20 mM Tris-HCl pH 7.4, 150 mM NaCl, 10% glycerol, 0.5 mM EDTA, 5 mM MgCl2, 0.05% Triton X-100, 0.1% Tween, and PMSF) and bound proteins were eluted with 4 bead volumes of B015 containing 0.2 mg/mL of FLAG peptide (Sigma) for 1 hr. The FLAG affinity purified complexes were further immunopurified by affinity chromatography using 10 μl of anti-HA conjugated agarose beads (Santa Cruz). After incubation for 4 hr, HA beads were washed 4 times with 800 μL of buffer B015 in spin columns (Pierce, 69702) and eluted under native conditions using HA peptide (Roche). Ten percent of the eluate was resolved on SDS-PAGE and Sylver stained using the silverquest kit (from invitrogen). The remaining material was stained with Coomassie-R250 and subsequently analysed by mass spectrometry at the Taplin Biological Mass Spectrometry facility (Harvard Medical School, Boston, Mass.).

In order to isolate MAML1-associated proteins in the presence or absence of activated Notch1, two-step affinity purification was performed on nuclear extracts from 4×10⁹ SupT1 cells stably expressing FLAG-HA tagged MAML1 treated for 8 hr with DMSO or GSI, followed by western blot analysis. Reciprocal immunoprecipitations of tagged-proteins were performed on Dignam nuclear extracts derived from SupT1 stably expressing: HA tagged-ICN1/FLAG-tagged MAML1 (4×10⁹ cells), HA tagged-ICN1/FLAG-tagged LSD1 (2×10⁹ cells), HA tagged-ICN1/FLAG-tagged PHF8 (2×10⁹ cells), HA tagged-ICN1/FLAG-tagged BRG1 (2×10⁹ cells) and control SupT1. After two step affinity chromatography, protein complexes containing both tagged-proteins were peptide eluted and analyzed by western blot. For endogenous protein immunoprecipitations, nuclear extracts (500 μg-1 mg) were incubated with antibodies (1-2 μg) for 4 hr, followed by addition of 10 μL protein G Sepharose beads (Fast flow, Sigma) for 45 min before washing five times with 800 μL of buffer B015 in spin columns (Pierce).

Chromatin Immunoprecipitation Assays (ChIP):

For ChIP experiments, 6×10⁷ cells were cross-linked for 10 min with 1% formaldehyde (sigma) at room temperature. The cross-linking reaction was stopped by adding glycine to a final concentration of 0.125 M for 10 min at room temperature. Cells were washed twice with cold PBS and incubated on ice for 7 min in 2 mL of buffer containing 15 mM Tris-HCl (pH 7.4), 0.3 M sucrose (sigma), 60 mM KCl, 15 mM NaCl, 5 mM MgCl2, 0.1 mM EGTA and 0.1% NP-40. Each cell suspension was then layered over 8 mL sucrose cushion (15 mM Tris-HCl, 1.2 M sucrose, 60 mM KCl, 15 mM NaCl, 5 mM MgCl2 and 0.1 mM EGTA) and centrifuged at 10.000 g for 20 min at 4° C. Nuclear pellet was lysed with 1 mL lysis buffer (50 mM Tris-HCl pH 8, 10 mM EDTA, 1% SDS) complemented with Protease Inhibitor Cocktail (Roche). Chromatin was sonicated to generate DNA-fragments of approximately 300 to 500 bp in an ultra sonicator water bath (Bioruptor, Diagenode) using ten cycles of 30 s/on and 30 s/off. After centrifugation at 13.000 g for 20 min, an aliquot of sonicated DNA was reverse-crosslinked by addition of 250 mM NaCl and incubation at 65° C. for 6 h. DNA was extracted by phenol-chloroform, quantified using nanodrop and run on a 1% agarose gel to confirm DNA fragment size. The antibodies were pre-bound to Invitogen Dynal magnetic beads (Protein A or G beads) in PBS containing BSA (5 mg/mL) and chromatin was pre-cleared with beads for 4 h at 4° C. Immunoprecipitation was performed using 20 μg of chromatin and 2-3 μg of antibody coupled to 15 μL of beads in ChIP buffer (20 mM Tri-HCl pH 8, 150 mM NaCl, 2 mM EDTA, and 1% Triton X-100) complemented with Protease Inhibitor Cocktail. After overnight incubation at 4° C., beads were washed 4 times with wash buffer 1 (20 mM Tri-HCl pH 8, 150 mM NaCl, 2 mM EDTA, 1% Triton X-100 and 0.1% SDS) and 4 times with wash buffer 2 (20 mM Tri-HCl pH 8, 500 mM NaCl, 2 mM EDTA, 1% Triton X-100 and 0.1% SDS) using the DynaMag-2 magnet (Invitogen). Elution of immunoprecipitated DNA was performed in buffer containing 1% SDS and 100 mM NaHCO3. Crosslinking was reversed by incubation at 65° C. and proteins were degraded by addition of proteinase K (Sigma). Eluted DNA and 10% of input DNA were purified using phenol-chloroform extraction followed by isopropanol precipitation or using QIAquick PCR purification (Qiagen), according to the manufacturer instructions. Resultant DNA was dissolved in 60 μL of water containing 10 mM Tris-HCl pH 8. ChIP DNA was analysed by SYBR Green quantitative PCR (Qiagen) using specific primers. qPCR was carried out in the LightCycler480 (Roche) with a 15 min DNA denaturation step at 95° C., followed by 40 cycles of 15 s at 95° C., 30 s at 58° C. and 30 s at 72° C. PCR measurements were performed in duplicate. The average of the technical replicates was normalized to input DNA per set of primer using the comparative CT method (2−ΔΔCT). Averages and standard deviations of the biological replicate values are shown in the figures. The number of biological replicates is indicated in the figure legends.

Western Blots:

Cells were lysed in lysis buffer (50 mM Tris-HCl, 120 mM NaCl, 5 mM EDTA, 0.5% NP-40 and PMSF) and briefly sonicated. Cell lysates and immunoprecipitates were boiled in SDS sample buffer and resolved on a 7% SDS-PAGE gel (Biorad). Proteins were liquid-transferred (Biorad) to nitrocellulose membrane in transfer buffer (20% methanol, 25 mM Tris, 192 mM Glycine, 0.037% SDS) during 90 min at 100V.

Quantitative RT-PCR:

Total RNA was isolated using Trizol reagent (Invitrogen) and reverse transcription was performed with 500 ng of RNA using SuperScript II (Invitrogen) and oligo-dT, according to the manufacturer's instructions. PCR measurements were performed in duplicate using SYBR Green (Qiagen). Amplification was carried out in the LightCycler480 (Roche) with a 15 min DNA denaturation step at 95° C., followed by 40 cycles of: 15 s at 95° C., 30 s at 60° C. and 30 s at 72° C. The average of the technical replicates was normalized to GAPDH levels using the comparative CT method (2−ΔΔCT). Averages and standard deviations of at least 3 experiments are shown in the figures.

Quantification of Nascent Transcripts:

RNAs were isolated using the Trizol reagent (Invitrogen) and treated with DNase (M610A promega) for 30 min at 37° C. The reaction was stopped according to the manufacturer's instructions and reverse transcription was performed with 1 μg of RNA using SuperScript II (Invitrogen) and random primers. PCR measurements were performed as described above (Q-RT-PCR) using intronic primers.

Notch-Responsive Reporter Assay:

HeLa cells were co-transfected with 1 μg of a Notch-responsive firefly luciferase reporter containing 6 CSL-binding sites (p6XCBS-luc), 100 ng of TK-Renilla-luciferase vector (transfection control) and various amount (0.1-0.3-1 μg) of the MigR1-ICN1 expression vector. Transfections were performed in 6-wells plates using JetPEI reagent (Polyplus) according to manufacturer's instruction. Firefly luciferase activity was measured 24 hours post-transfection and normalized to Renilla luciferase expression. The values in the figures are Relative Luciferase Units (RLU) represented as fold induction over the luciferase activity measured in the absence of ICN1 (cells transfected with p6XCBS-luc and an empty vector). The mean and standard deviations from several experiments are shown in the figures. The number of experiments is indicated in the figure legends.

Subcutaneous Xenograft Tumor Model:

Female SCID mice (C.B.-17/IcrHan™Hsd-Prkdcscid) were obtained from Harlan Laboratories (Gannat, France). Animals were maintained in specific pathogen-free animal housing at the Center for Exploration and Experimental Functional Research (CERFE, Evry, France) animal facility. The human T-ALL cell line SupT1 was infected with retroviral vectors encoding shRNA directed against human PHF8, CSL, and LSD1, or a control shRNA. 72 hours post-infection, cells were selected with 2 μg/mL puromycin for 72 hours. At this point, the cells were maintained in fresh media for 2 days prior to injection into animals. Prior injection, cells were washed and resuspended in DMEM: 50% Matrigel (BD Pharmingen). 5×10⁶ cells (in 1004) were injected to each mouse by subcutaneous route in the right flank (n=5 per group). Tumor volume was evaluated by measuring tumor diameters, with a calliper, three times a week during the follow-up period (23 days). The formula TV (mm³)=[length (mm)×width (mm)2]/2 was used, where the length and the width are the longest and the shortest diameters of the tumor, respectively.

Flow Cytometry, Cell Proliferation and Cell Cycle Analysis:

The following antibodies were used for flow cytometry: CD127-PE clone R34.34 and the IgG1-PE (from Beckman Coulter). Flow cytometry was performed on a BD FACSCalibur or MACSQUANT cytometer (Miltenyi). For cell proliferation assays, cells were plated at 2×105/mL in triplicate. Proliferation of shRNA-transduced T-ALL cells was followed by cell counting using the MACSQUANT cytometer (gated on live cells). Flow-cytometric cell cycle analysis was performed by staining DNA content of T-ALL cell lines using DAPI. SupT1 cells were also analyzed with EdU-DAPI staining to precisely define the percentage of cells at the G0/G1 phase. Briefly, SupT1 expressing control, CSL, PHF8 or LSD1 shRNAs were treated with 10 μM EdU for 2 hrs. Cells were washed with PBS and fixed with PBS-4% PFA for 10 minutes at room temperature. After permeabilization, EdU incorporation was detected following the manufacturer's instructions (Click-iT, invitrogen) and total DNA content was measured using DAPI.

Nuclear Run-On:

SupT1 cells expressing control or AF4p12 shRNA were harvest, washed twice with cold PBS and incubated on ice for 7 min in 2 mL of buffer containing 15 mM Tris-HCl (pH 7.4), 0.3 M sucrose (sigma), 60 mM KCl, 15 mM NaCl, 5 mM MgCl2, 0.1 mM EGTA and 0.1% NP-40. Cell suspension was layered over 8 mL sucrose cushion (15 mM Tris-HCl, 1.2 M sucrose, 60 mM KCl, 15 mM NaCl, 5 mM MgCl2 and 0.1 mM EGTA) and centrifuged at 10.000 g for 20 min at 4° C. Nuclei were resuspended in freezing buffer (50 mM Tris-HCl pH=8, 40% glycerol, 5 mM MgCl2 and 0.1 mM EDTA) at a concentration of 5×106/mL and freezed (at −80° C.). Nuclear Run-on assays were performed as described previously (Core et al., 2008), except that we used 5×10⁵ nuclei per reaction containing 500 μM ATP, CTP, GTP and br-UTP and 0.5% sarkosyl. The reaction was performed at 30° C. for 5 minutes. RNAs transcribed during the assay were purified using anti-BrdU beads (Santa cruz) and reverse transcription was performed using SuperScript II (Invitrogen) and random primers. PCR measurements were performed as described above (Q-RT-PCR) using intronic primers.

In Vitro T-Cell Differentiation:

CD34+ cells from human umbilical cord blood samples were sorted (Stemcell technologies) (≧95% purity) and cultured on OP9-DL1 stromal cells in αMEM media containing FLT3L (5 ng/ml), and IL7 (5 ng/ml) for 16 days. At day 16, most progenitors (˜90%) were engaged to the T-cell lineage (pre-T cells) as determined by the expression of CD5 and CD1a. pre-T-cells were cultured for 2 additional days on OP9-DL1 cells in presence or absence of GSI (5 μM).

Results

Identification of NOTCH1-Associated Cofactors:

Mutational activation of NOTCH1 leading to aberrant ICN1 production and translocation into the nucleus is a frequent event in T-ALL. Beside CSL and MAML1, nuclear partners that support NOTCH1 transcriptional program and tumorigenesis remain to be determined. To identify such factors, we purified ICN1 from SupT1 cells, a human NOTCH1-dependent T-ALL cell line. We generated stable SupT1 cells expressing human ICN1 tagged with both Flag and HA epitopes (F/H-ICN1). Western blot (WB) assay showed that tagged ICN1 is expressed in the nucleus at a level comparable to that of endogenous ICN1. Importantly, F/H-ICN1 was able to restore Notch-responsive gene expression and Notch-dependent proliferation after inhibition of endogenous Notch using g-secretase inhibitor (GSI), indicating that F/H-ICN1 is functional.

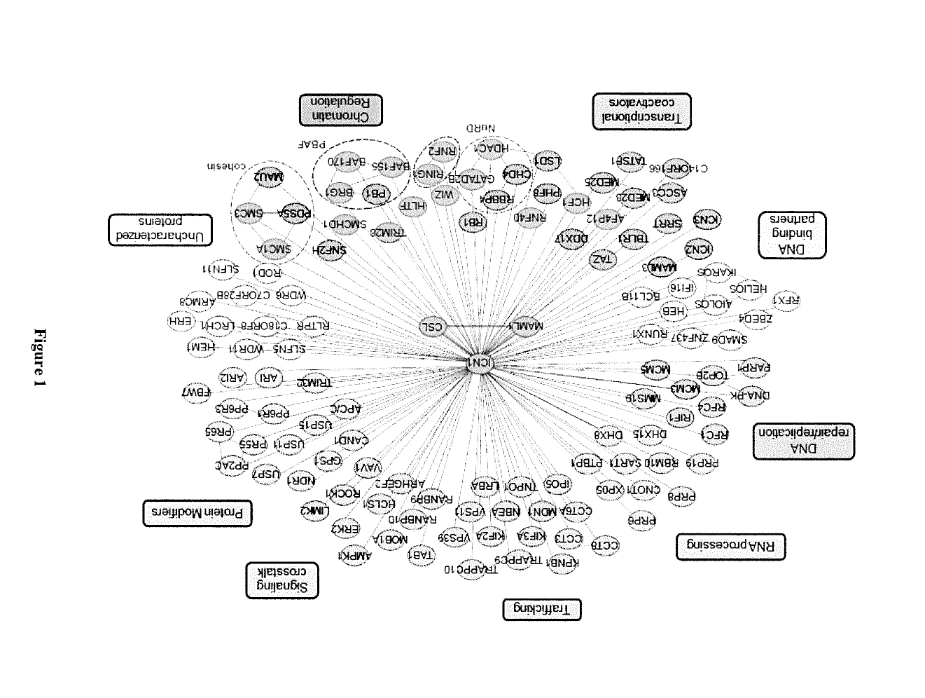

F/H-ICN1 and its associated partners were purified from nuclear extracts derived from SupT1 cells using tandem affinity chromatography. Mass spectrometry (MS) analysis identified protein-partners of ICN1 (FIG. 1). Major MS-identified ICN1 nuclear partners are the core components of the Notch-activation complex: MAML1 and CSL. The numbers of recovered peptides and the intensity of the corresponding silver-stained bands indicate that MAML1 and CSL are stoichiometric ICN1-partners. Other Notch pathway components such as MAML3, ICN2 and ICN3 were also recovered. Importantly, 27 out of 127 interacting proteins have been previously associated with Notch signaling. Finally, some of ICN1-partners were tested and confirmed by WB. Taken together, these results validate the ICN1-purification strategy.

Interaction network analysis of ICN1 partners revealed several functional classes of proteins (FIG. 1). These include protein-modifying enzymes with a potential role in NOTCH1 regulation such as the tumor suppressor FBW7 known to target ICN1 for ubiquitination and degradation (Aifantis et al., 2008), as well as deubiquitinating enzymes, kinases and phosphatases. ICN1 associates with several lineage-specific transcription factors that comprise major regulators of T-cell development (BCL11B, HEB and RUNX1) and IKAROS family members, including IKZF1, a tumor suppressor that represses Notch transcriptional responses. Moreover, interactions between ICN1 and components of other signaling pathways, such as the MAP kinase family member ERK2 and the TGFβ/BMP signaling mediator SMAD9, were also detected. Importantly, among ICN1-associated proteins we found numerous regulators of gene expression that act at various steps of transcriptional activation (Table 1). These encompass well-characterized coactivators, such as HCF1, ASCC3 and subunits of the Mediator complex, as well as proteins with putative functions in transcriptional regulation, for example the MLL-fusion partner AF4p12 (also known as FRYL), a poorly-characterized protein that exhibits transcriptional activation properties (Hayette et al., 2005). Chromatin-modifying enzymes, including the PBAF nucleosome-remodeling complex, RNF40 (a subunit of the E3 ubiquitin-ligase BRE1 that monoubiquitinates H2B) and the histone demethylases LSD1 and PHF8, were also recovered.

Thus, ICN1 interacts with a varied set of proteins in T-ALL cells that could reflect the diversity of Notch functions and regulation. In the present study, we focused on the characterization of ICN1-cofactors important for its transcriptional activity.

All the NOTCH1-associated cofactors involved in transcription which have been identified in the present study are reported in Table 1:

TABLE 1
Identified Notch partners (ICN1 partners) involved in transcription

| | | | |
|---|---|---|---|
| | | Role in transcriptional | |
| Protein | Primary function | activation | References |

Transcriptional activators

| | | | |
|---|---|---|---|
| BRG1 | Nucleosome | Components of the PBAF | (Ho and Crabtree, |
| PB1 | remodeling | chromatin remodeling complex. | 2010) |
| BAF170 | | PBAF regulates transcription by | |
| BAF155 | | altering the chromatin structure. | |
| RNF40 | Histone H2B | Monoubiquitination of H2B- | (Osley, 2006; Zhu |
| | ubiquitin ligase | K120 is a prerequisite for the | et al., 2005) |
| | | methylation of H3K4 (initiation) | |
| | | and H3K79 (elongation). | |
| LSD1 | Histone | Activates transcription by | (Garcia-Bassets et |
| | demethylase | demethylating the repressive | al., 2007; Metzger |
| | | mark H3K9me1/2 and non- | et al., 2005; Perillo |
| | | histone proteins (such as the | et al., 2008; Sakane |
| | | HIV-1 transactivator Tat). | et al., 2011) |
| PHF8 | Histone | Activates transcription by | (Feng et al., 2010; |
| | demethylase | removing multiple repressive | Fortschegger et al., |
| | | marks including H3K9me1/2, | 2010; Horton et al., |
| | | H3K27me2 and H4K20me1. | 2010; Kleine- |
| | | | Kohlbrecher et al., |
| | | | 2010; Liu et al., |
| | | | 2010b; Loenarz et |
| | | | al., 2010; Qi et al., |
| | | | 2010; Qiu et al., |
| | | | 2010; Zhu et al., |
| | | | 2010) |
| TBLR1 | Corepressor/coactivator | Mediates the exchange of | (Perissi et al., |
| | exchange factor | corepressor for coactivator | 2004; Perissi et al., |
| | | during activation by signal- | 2008) |
| | | dependent transcription factors | |
| MED23 | Transcriptional | Components of the mediator | (Malik and Roeder, |
| MED25 | initiation | complex, which promotes the | 2005) |
| | | assembly of RNA polymerase II | |
| | | and general transcription factors. | |
| C14ORF166 | RNA PolII | Interacts with RNA polymerase | (Perez-Gonzalez et |
| | regulation | II and positively regulates its | al., 2006) |
| | | activity. | |
| TATSF1 | Transcriptional | Couple transcription elongation | (Li and Green, |
| | elongation | to RNA processing. | 1998; Zhou and |
| | | | Sharp, 1996) |
| HCF1 | Transcriptional | Transcriptional coactivator for | (Kristie and Sharp, |
| | coactivator | multiple cellular and viral | 1993; Vogel and |
| | | transcription factors. | Kristie, 2000) |
| TAZ | Transcriptional | Transcriptional coactivator in | (Liu et al., 2011) |
| | coactivator | the Hippo signaling pathway | |
| ASCC3 | Transcriptional | Helicase that unwind duplex | (Dango et al., |
| | activator | DNA. Play an essential role in | 2011; Jung et al., |
| | | transcriptional activation by | 2002) |
| | | various transcription factors. | |
| AF4P12 | Uncharacterized | Exhibits transcriptional | (Hayette et al., |
| | | activation properties | 2005) |
| NOTCH2 | Notch paralogues | Heterodimerization between | (Nam et al., 2007) |
| NOTCH3 | | Notch paralogues may play a | |
| | | role in the regulation of Notch1 | |
| | | activity. | |

Factors involved in transcription

| | | | |
|---|---|---|---|
| SMC1A | Cohesin complex. | Cohesin facilitates | (Fay et al., 2011; |
| SMC3 | Involved in | transcriptional activation by | Kagey et al., 2010; |
| PDS5A | chromosome | promoting enhancer-promoter | Pauli et al., 2010; |
| MAU2 | cohesion during cell | communication. | Seitan et al., 2011) |
| | cycle | | |
| AMPK | Metabolic pathway | Stimulates transcriptional | (Bungard et al, |
| | kinase | elongation by directly | 2010) |
| | | phosphorylating histone H2B at | |
| | | serine 36. | |
| ERK2 | Signaling pathway | Upon MAPK pathway | (Agoulnik et al., |
| | kinase | activation, ERK2 phosphorylates | 2008; Chen et al., |
| | | and activates transcription | 2007; Madak- |
| | | factors. Component of hormone | Erdogan et al., |
| | | receptors activation complex. | 2011; Vicent et al., |
| | | | 2006) |
| DNAPK | DNA repair | DNA-PK complex induces DNA | (Abramson et al., |
| TOP2B | | double-strand breaks required | 2010; Haffner et |
| PARP1 | | for transcription activation by | al., 2010; Ju et al., |
| | | various transcription factors. | 2006; Nock et al., |
| | | | 2009; Tyagi et al., |
| | | | 2011; Wong et al., |
| | | | 2009) |
| RANBP9 | Ran-GTPase | Essential for transcriptional | (Harada et al., |
| RANBP10 | binding partners | activation by nuclear hormone | 2008; Poirier et al., |
| | | receptors. | 2006; Rao et al., |
| | | | 2002) |
| MMS19 | DNA repair | Interacts with the estrogen | (Wu et al., 2001) |
| | | receptor and stimulates its | |
| | | transcriptional activity | |
| MCM5 | DNA replication | Directly interacts with STAT1 | (Snyder et al., |
| | | and regulates interferon-induced | 2005; Zhang et al., |
| | | gene expression. | 1998) |
| SRRT | RNAi pathway | Recently reported to directly | (Andreu-Agullo et |
| | component | activate transcription. | al., 2012) |
| DDX17 | RNA helicase | DDX17 acts as transcriptional | (Watanabe et al., |
| | | coactivators for several | 2001; Wortham et |
| | | transcription factors (such as | al., 2009) |
| | | estrogen receptor) | |
| PRP19 | mRNA splicing | In Saccharomyces cerevisiae, | (Chanarat et al., |
| | | Prp19 acts as a transcription | 2011) |
| | | elongation factor. | |
| ERH | Uncharacterized | Highly conserved, exhibits | (Wan et al., 2005) |
| | | transcriptional regulation | |
| | | activities | |
| USP7 | Ubiquitin hydrolase | Regulates transcription by | (van der Knaap et |
| | | histone H2B deubiquitylation | al., 2005) |

The PBAF Complex, LSD1, PHF8 and AF4p12 Associate with ICN1:

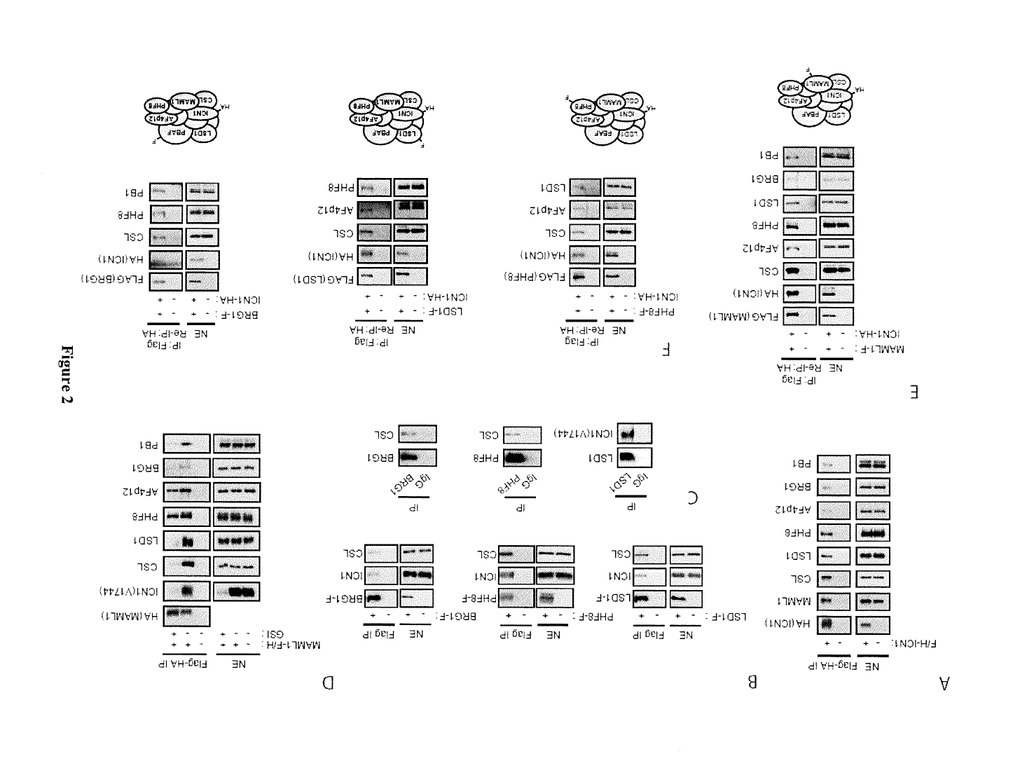

Western Blot analysis of ICN1 purified material confirmed the interaction with PBAF subunits BRG1 and PB1, the histone demethylases LSD1 and PHF8, and AF4p12 (FIG. 2A). Importantly, endogenous ICN1 and CSL were immunoprecipitated with Flag-tagged LSD1, PHF8 or BRG1 (FIG. 2B). Moreover, immunoprecipitation of endogenous LSD1, PHF8 and BRG1 showed a specific interaction with components of the Notch-activation complex, as revealed by the presence of endogenous ICN1 or CSL (FIG. 2C), suggesting that PBAF, LSD1 and PHF8 are part of the Notch-activation complex. In agreement with these observations, WB analysis of MAML1-interacting proteins purified from SupT1 nuclear extracts confirmed its association with the newly identified cofactors (FIG. 2D). Interestingly, GSI treatment severely altered MAML1 association with CSL, LSD1, PB1 and BRG1, indicating that these interactions are Notch-dependent. However, binding of PHF8 and AF4p12 to MAML1 was only marginally reduced (FIG. 2D). This suggests that MAML1 might recruit PHF8 and AF4p12 to the Notch-activation complex, while LSD1 and the PBAF complex could be ICN1 or CSL partners. Alternatively, recruitment of cofactors, including LSD1 and PBAF, may result from conformational changes induced by ICN1-CSL-MAML1 ternary complex formation.

Notch1-Associated Factors Assemble into a Single Complex Containing ICN1-CSL-MAML1:

To further confirm that Notch cofactors are integral components of the Notch-activation complex, nuclear extracts from SupT1 cells stably expressing Flag-MAML1 and HA-ICN1 were subjected to sequential immunopurifications using anti-Flag followed by anti-HA beads. Because MAML1 incorporation into the complex requires a contact with both ICN1 and CSL (Nam et al., 2006; Wilson and Kovall, 2006), this approach allowed us to isolate factors associated with the ICN1-CSL-MAML1 ternary complex. WB assay of the purified material revealed the presence of ICN1 cofactors (FIG. 2E), indicating that BRG1, PB1, LSD1, PHF8 and AF4p12 are physical partners of the Notch-activation complex.

To determine whether these factors form one or distinct ICN1-CSL-MAML1-containing complexes, we stably coexpressed HA-ICN1 and Flag-tagged PHF8, LSD1 or BRG1 in SupT1. Reciprocal immunoprecipitations detected the association of CSL with the purified PHF8-ICN1-, LSD1-ICN1- and BRG1-ICN1-containing complexes (FIG. 2F). Importantly, WB analysis also revealed the presence of LSD1 and AF4p12 in the PHF8-ICN1 purification, suggesting that these factors exist in a single complex. Furthermore, AF4p12 and PHF8 coimmunoprecipitated with LSD1-ICN1 complex, and both PHF8 and PB1 were detected in BRG1-ICN1 purification (FIG. 2F). These results indicate that PBAF, LSD1, PHF8 and AF4p12 associate in a single multifunctional complex containing ICN1-CSL-MAML1. Of note, our analysis does not exclude that other coactivators including those identified by MS analysis (Table 1) are also components of this complex, and it remains possible that Notch might form different complexes with other transcriptional regulators.

NOTCH1 cofactors are recruited to Notch-target genes:

To investigate the functional relevance of this newly characterized ICN1-associated complex, we addressed its role in Notch-mediated transcriptional activation. First, we studied the recruitment of PBAF, LSD1, PHF8 and AF4p12 to known Notch-target genes in immature T-cells (HES1, DTX1, IL7R, NOTCH3 and CR2). ICN1-binding sites were determined based on the recently published genome-wide map of NOTCH1 occupancy in T-ALL cell lines (Wang et al., 2011), and further validated by chromatin immunoprecipitation assay (ChIP) in SupT1. Chromatin prepared from SupT1 cells treated with DMSO or GSI was subjected to ChIP using the indicated antibodies. Consistent with the biochemical interactions (FIG. 2), MAML1, CSL, the PBAF complex (as revealed by BRG1 and PB1 binding), PHF8, LSD1 and AF4p12 were found associated with ICN1-binding sites. Importantly, GSI treatment, which released ICN1 and MAML1 from chromatin, reduced the recruitment of PBAF, PHF8 and AF4p12, but did not significantly affect CSL and LSD1 binding. Accordingly, a link between LSD1 and CSL-mediated repression has been reported (Di Stefano et al., 2011; Mulligan et al., 2011; Wang et al., 2007), supporting the idea that LSD1 occupancy of Notch-target genes after GSI treatment might be mediated by its association with CSL. These results suggest that ICN1, MAML1, PBAF, PHF8 and AF4p12 are recruited to CSL-binding sites after Notch signaling activation and may associate with resident LSD1 to form a functional Notch-activation complex.

PBAF, LSD1, PHF8 and AF4p12 are Required for Notch Transcriptional Activity:

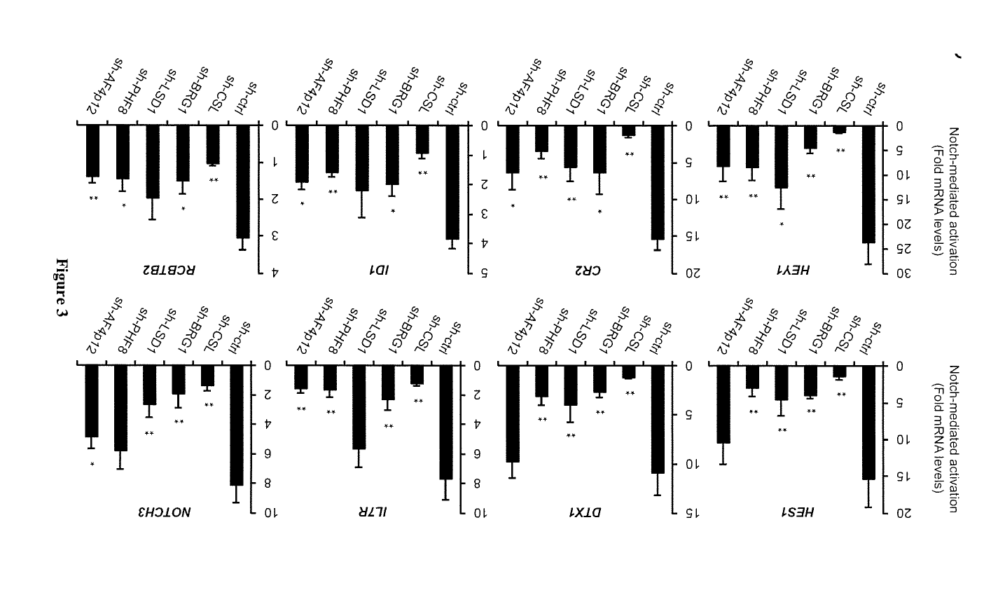

We next asked whether Notch-cofactors are required for its transcriptional activity. Efficient depletion of BRG1, LSD1, PHF8 and AF4p12 in SupT1 cells did not affect ICN1 levels. The eight Notch-responsive genes analyzed (i.e. HES1, DTX1, IL7R, HEY1, NOTCH3, CR2, ID1 and RCBTB2) were all found to be directly bound by NOTCH1 and positively regulated by Notch signaling in several T-ALL cell lines (Wang et al., 2011), including SupT1. As expected, CSL depletion completely abolished Notch transcriptional activity as measured by quantitative RT-PCR (FIG. 3). Knockdown of the ATPase subunit of PBAF remodeling complex, BRG1, strongly reduced ICN1-mediated transcriptional activation of all tested genes. Moreover, knockdown of LSD1, PHF8 or AF4p12 altered the expression of Notch-responsive genes, although some targets showed a differential sensitivity to LSD1, PHF8 or AF4p12 depletion (FIG. 3). This selective requirement might depend on gene-specific features, including the chromatin context and other regulatory sequences, rather than differences in the ability of ICN1 to mediate their recruitment.

IL7R expression at the cell surface plays a key role in Notch-induced T-cell development and leukemia (Magri et al., 2009) (Gonzalez-Garcia et al., 2009). Thus, we analyzed several human Notch-dependent TALL cell lines that constitutively express IL7R at the cell surface (SupT1, HPB-ALL, TALL1 and DND41). We found that disruption of Notch signaling by CSL depletion severely impaired IL7R expression in SupT1, HPB-ALL and TALL1, but not DND41 cells. Consistently, knockdown of Notch-cofactors resulted in a down-regulation of IL7R in SupT1, HPB-ALL and TALL1, but did not affect Notch-independent expression of IL7R in DND41 cells. This suggests that PHF8, LSD1 and AF4p12 are not general regulators of IL7R expression, but are specifically required for Notch-mediated regulation of IL7R. Overall, these results indicate that the newly identified cofactors of the Notch-activation complex control the expression of Notch-responsive genes.

AF4p12 is a Notch Transcriptional Coactivator:

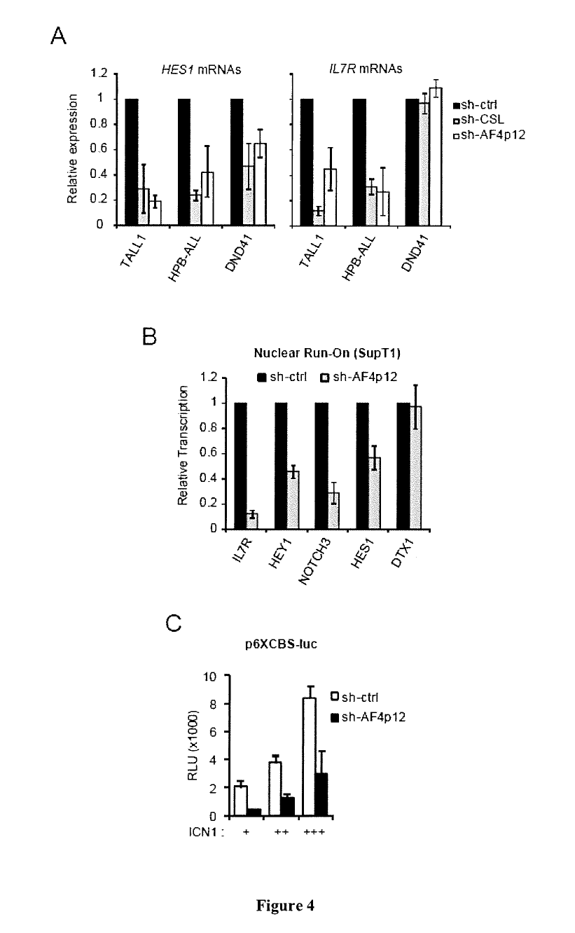

The function of AF4p12 in transcription remains largely uncharacterized (Hayette et al., 2005). Decreased expression of Notch-target genes, including HES1 and IL7R, was observed after depletion of AFp12 in several T-ALL cell lines (FIG. 4A). To exclude any defect in cotranscriptional RNA processing or alteration in mRNA stability, we first measured the levels of nascent pre-mRNAs using intronic primers. AF4p12 knockdown caused a severe decrease in pre-mRNA levels of several Notch-responsive genes, suggesting that AF4p12 affects the rate of transcription. To test this, we isolated nuclei from SupT1 cells expressing control or AF4p12 specific shRNA and performed nuclear run-on experiments. Analysis of transcripts generated during the run-on indicates that AF4p12 positively regulates the transcription of Notch target genes (FIG. 4B). Additionally, ICN1-mediated activation of a transiently transfected Notch-responsive luciferase reporter (p6XCBS-luc) was reduced by AF4p12 depletion (FIG. 4C) supporting a role of AF4p12 in Notch transcriptional activity. Next, we determined the consequence of AF4p12 depletion on ICN1 and RNAPII recruitment to Notch-target genes using ChIP assay. While knockdown of AF4p12 had no effect on ICN1 recruitment, it reduced the recruitment of RNAPII to NOTCH3 and IL7R locus. Consistent with the absence of a role for AF4p12 in ICN1-mediated transcription of DTX1 in SupT1 (FIG. 4B), AF4p12 depletion did not affect RNAPII recruitment to the DTX locus. Taken together, our results show that AF4p12 acts as a Notch coactivator and plays a role in transcriptional activation events subsequent to ICN1 recruitment that are required for RNAPII assembly at several Notch-responsive loci.

LSD1 is a Component of the CSL-Repressor Complex and Notch-Activation Complex:

In line with recent reports (Wang et al., 2007) (Mulligan et al., 2011), the results here suggest that LSD1 associates with the CSL-repressor complex. Additionally, our results demonstrate that LSD1 is an integral component of the Notch-activation complex (FIG. 2). We therefore reasoned that LSD1 might play a dual role in Notch signaling. First, we examined LSD1 binding to NOTCH3, CR2 and HES1 genes in SupT1 cells expressing control or CSL-specific shRNA. While Notch inactivation by GSI did not significantly affect LSD1 binding, knockdown of CSL reduced LSD1 occupancy regardless of Notch activation. These results support a model in which LSD1 occupies Notch-target genes as part of the CSL-repressor complex and the Notch-activation complex. In both cases, LSD1 recruitment is CSL dependent. Next, we performed coimmunoprecipitation experiments in SupT1 cells treated with DMSO or GSI. While LSD1 binding to endogenous MAML1 and ICN1 was strongly reduced by GSI, CSL coimmunoprecipited with LSD1 under both conditions. Thus, LSD1 can interact with CSL independently of ICN1. Consistently, endogenous LSD1 and CSL interact in U937 cells, a myeloid cell line that does not express a constitutively active NOTCH1. Moreover, CSL knockdown in U937 cells reduced LSD1 recruitment to the HES1 promoter and HEY1 enhancer indicating that LSD1 is a component of the DNA-bound CSL-repressor complex.

LSD1 is Required for CSL-Mediated Repression of Notch-Target Genes:

We next determined the role of LSD1 in CSL-mediated repression. Similar to CSL knockdown, depletion of LSD1 in U937 cells resulted in a significant increase of HES1, HEY1 and DTX1 mRNA levels. Moreover, treatment of U937 cells with LSD1 inhibitors, tranylcypromine (TCP) and S2101, also increased HES1, HEY1 and DTX1 expression. A similar result was obtained in the monocytic cell line THP1 that does not express a constitutively active NOTCH1. Importantly, inhibition of LSD1 by either shRNA or S2101 treatment in GSI-treated SupT1 cells resulted in a depression of Notch-responsive genes, while the expression of the control S14 gene was not affected. LSD1 represses transcription through H3K4me1/me2 demethylation (Shi et al., 2004). Therefore, we tested whether LSD1 depletion is associated with an increase of H3K4 methylation at Notch-target genes. While H3K4me1 and H3K4me3 were not significantly affected, we observed a significant increase in H3K4me2 levels at Notch target loci after LSD1 depletion in U937 cells. These results indicate that in the context of the CSL-repressor complex, LSD1 contributes to Notch-target genes repression by removing the activating H3K4me2 mark.

A Functional Switch of LSD1 Activity Control Notch-Target Genes Activation:

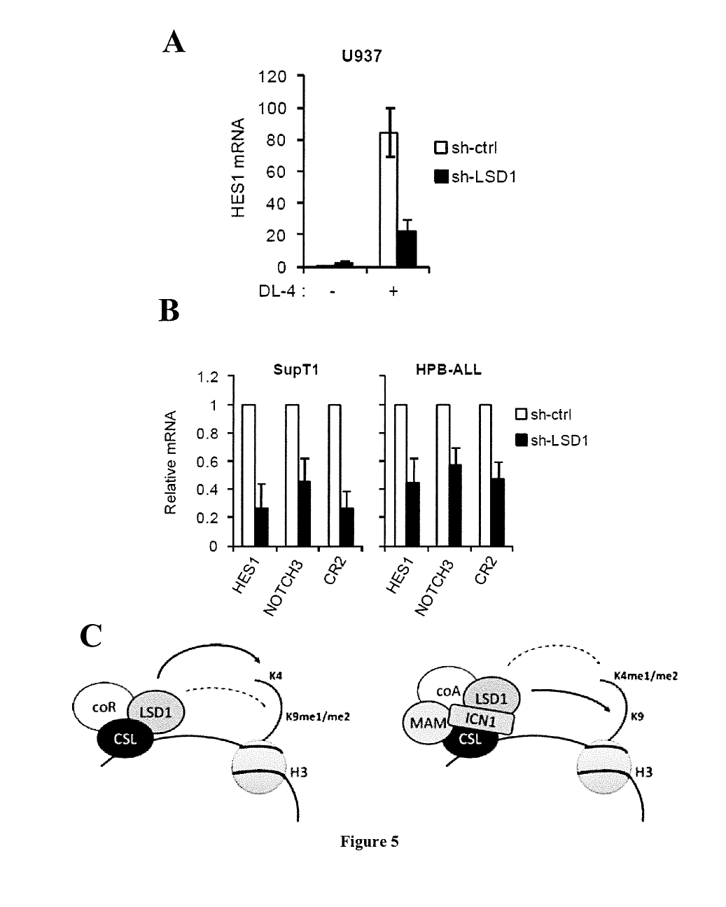

LSD1 can either suppress or promote transcription depending on its substrate (Metzger et al., 2005). The results suggest a functional switch in LSD1 activity after Notch activation. To test this hypothesis, we cultured U937 cells expressing control or LSD1 specific shRNA in the presence of recombinant Delta-like 4 (DL-4) Notch ligand. LSD1 knockdown reduced Notch-induced HES1 transcription (FIG. 5A), without affecting ligand-mediated NOTCH1 cleavage or stability, suggesting a transcriptional inhibition downstream of ICN1 release. Consistently, both TCP and S2101 reduced HES1 activation, indicating that the demethylase activity of LSD1 is required for HES1 induction by Notch. Knockdown of LSD1 (Figure S4H-I) and TCP treatment impaired ICN1-induced activation of the Notch-responsive reporter (p6XCBS-luc) in HeLa cells. These data imply that LSD1, which acts as a transcription repressor in the absence of Notch, also functions as a Notch coactivator. In support of this conclusion, knockdown of LSD1 in human NOTCH1-dependent T-ALL cell lines impaired the expression of the several Notch-dependent genes, including HES1, NOTCH3 and CR2 (FIG. 5B). Importantly, inhibition of LSD1 activity by S2101 in T-ALL cells resulted in a dose-dependent repression of Notch-target genes.

The inventors next explored the mechanism underlying the functional requirement of LSD1 demethylase activity in Notch-dependent transcription. Knockdown of LSD1 in SupT1 reduced its binding to HES1 and CR2, but did not alter CSL binding or Notch-dependent MAML1 recruitment, indicating a block downstream ICN1-CSL-MAML1 binding. LSD1 is reported to function as a coactivator for several transcription factors through demethylation of H3K9me1/me2 repressive marks (Metzger et al., 2005). Accordingly, LSD1 depletion significantly increased H3K9me2 levels, but not H3K4 methylation, at HES1 and CR2 gene in DMSO treated SupT1 cells. In contrast, in GSI treated cells, knockdown of LSD1 did not significantly affect H3K9 methylation, but resulted in a specific increase in H3K4me2 levels. Taken together, our results suggest that a critical function of LSD1 in Notch-dependent transcription is to trigger H3K9me2 demethylation. However, in the absence of Notch, LSD1 acts preferentially on H3K4me2 and contributes to CSL-mediated repression (FIG. 5C).

PHF8 Demethylase Activity is Required for Notch-Mediated Activation of its Target Genes:

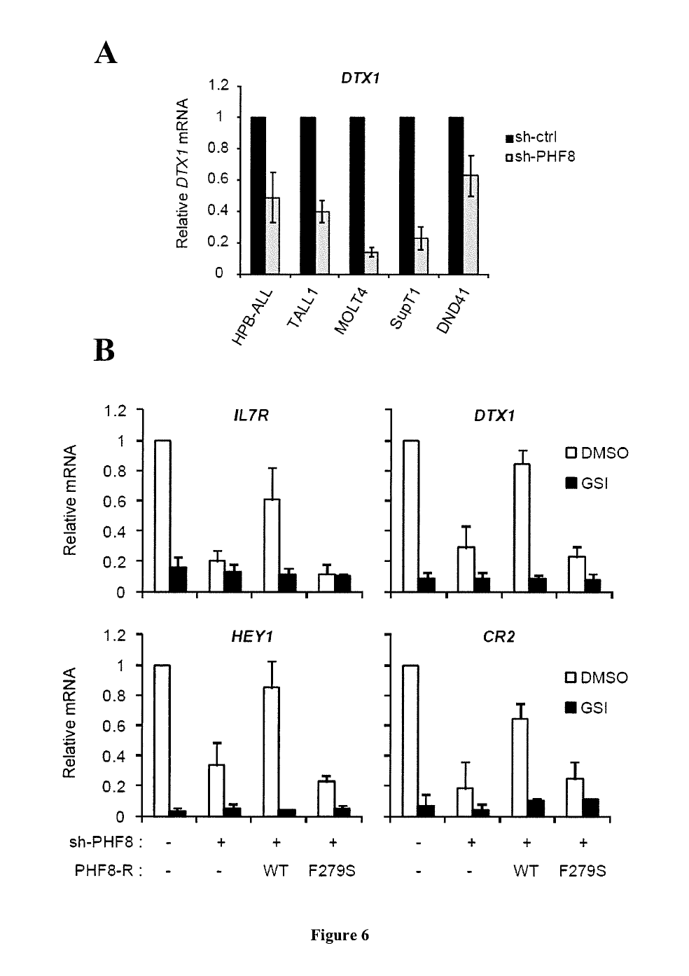

Consistent with the results obtained in SupT1 cells, knockdown of PHF8 in several T-ALL cell lines decreased the expression of Notch-responsive genes (FIG. 6A). Moreover, depletion of PHF8 in HeLa cells impaired ICN1-mediated activation of the p6XCBS-luc reporter, indicating that PHF8 acts as a transcriptional coactivator of Notch.

PHF8 removes multiple transcriptional repressive marks, including H3K9me1/me2, H4K20me1 and H3K27me2 (Horton et al., 2010; Liu et al., 2010b; Loenarz et al., 2010; Qi et al., 2010). To determine whether PHF8 demethylase activity is required for Notch-target genes activation, we performed rescue experiments in SupT1 by expressing shRNA-resistant PHF8 or the catalytically inactive F279S mutant. As shown in FIG. 6B, the expression of Notch-responsive genes was restored by wide-type PHF8 but not by the inactive mutant. These data indicate that PHF8 controls Notch transcriptional responses through its demethylase activity.

To investigate histone marks regulated by PHF8 at Notch target loci, we performed ChIP experiments in SupT1. Analysis of PHF8-binding at the IL7R locus and DTX1 locus showed that PHF8 peaks at ICN1-containing enhancers but also at the transcription start site (TSS), consistent with its ability to bind H3K4me3. In agreement with its function as a transcriptional coactivator, PHF8 depletion reduced H3K4me3 levels at the IL7R and DTX1 TSS. An increase of H3K9me1 levels, and to a lesser extent H3K9me2 levels, was observed only at the TSS-region (primer 2 for IL7R and DTX1), but not at ICN1-binding region (primer 4 for IL7R and primer 3 for DTX1). Moreover, loss of PHF8 did not lead to any detectable increase in H4K20me1 levels. In contrast, while PHF8 depletion did not affect total H3 level, it caused an accumulation of the repressive H3K27me2 mark at the two tested loci, suggesting that it is actively removing this mark. Consistently, upon knockdown of PHF8, we observed a robust increase of H3K27me2 levels, but not H3K27me3, at other Notch-target genes (including HES1, CR2 and NOTCH3). These results suggest that in the context of the Notch-activation complex, PHF8 may control Notch responses by removing H3K27me2. In agreement with this model, reduction in PHF8 recruitment after Notch signaling inhibition by GSI was accompanied by an increase of H3K27me2 mark at both IL7R and DTX1 loci.

LSD1 and PHF8 are Required for NOTCH1-Dependent T-ALL Cell Proliferation:

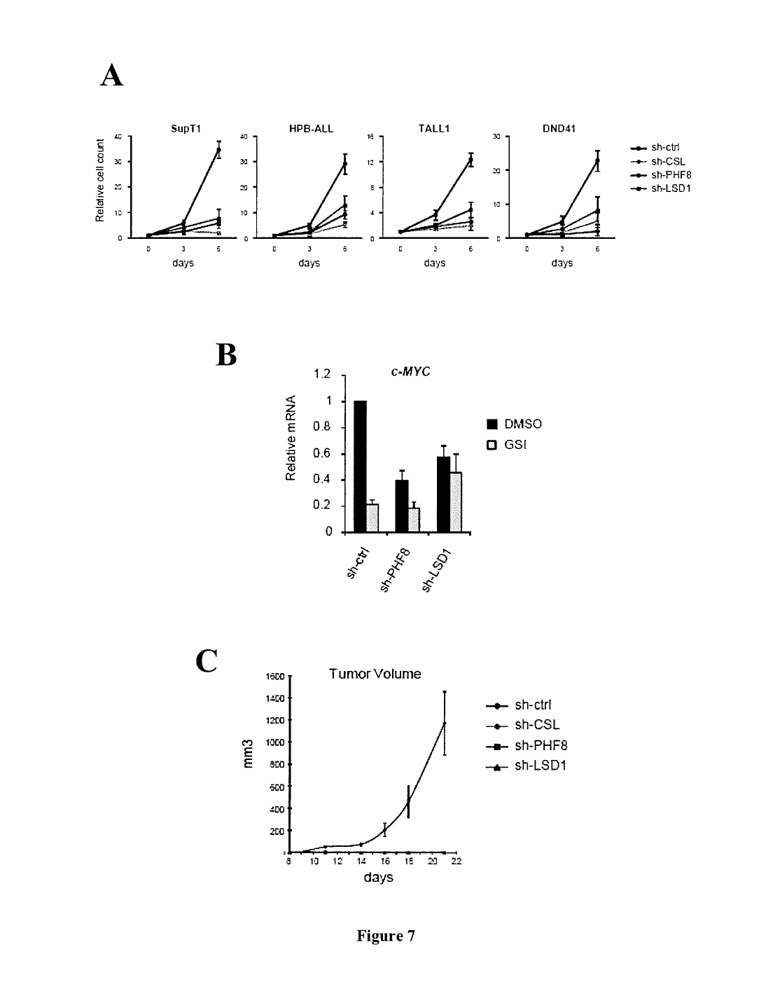

Previous studies have shown that inhibition of the Notch pathway induces cell-cycle arrest and alters growth capacities of NOTCH1-dependent leukemia cells (Weng et al., 2004). These observations prompted us to assess the role of LSD1 and PHF8 in NOTCH1 oncogenic functions. Abrogation of Notch signaling by depletion of CSL, PHF8 or LSD1 in a panel of T-ALL cell lines bearing activating NOTCH1 mutations (SupT1, HPB-ALL, TALL1 and DND41) resulted in a marked reduction in their proliferation (FIG. 7A). Importantly, knockdown of CSL, PHF8 or LSD1 induced a G0/G1 cell-cycle arrest in all NOTCH1-dependent T-ALL cells tested, but did not significantly affect cell-cycle progression in MOLT4 and H9, which are Notch-independent T-ALL cell lines (Weng et al., 2004). Depletion of PHF8 and LSD1 suppressed Notch-mediated expression of c-MYC (FIG. 7B), a gene that has a key role in tumorigenesis induced by NOTCH1, which further support their role in the oncogenic activity of Notch signaling. Consistent with this, T-ALL cells expressing CSL, PHF8 or LSD1 shRNAs did not establish tumors in a mouse xenograft model (FIG. 7C). These results indicate that LSD1 and PHF8 are key regulators of NOTCH1 oncogenic functions in T-ALL cells and support the idea that targeted therapies interfering with PHF8 or LSD1 activities could be useful in the treatment of cancers that have activated NOTCH1 alleles.

Notch Cofactors are Recruited to Notch-Target Genes During T-Cell Development:

Notch signaling pathway plays a key role during T-cell development. To investigate whether Notch cofactors identified in leukemic T cells are recruited to Notch-target genes during physiological T lymphopoiesis, we performed ChIP experiments using an in vitro T-cell differentiation system that allows analysis of early stages (Schmitt and Zuniga-Pflucker, 2002). Hematopoietic stem cells (HSCs) from human umbilical cord blood were cultured for 16 days on OP9-DL1 cells, a bone-marrow stromal cell line that ectopically expresses the Notch ligand DL1, then DMSO or GSI was added for 2 additional days. The coculture induced the expression of several Notch-target genes (including DTX1, NOTCH3 and IL7R) that was accompanied by differentiation and proliferation of the progenitors. At day 16 of culture, most precursors were committed to the T-cell lineage, as indicated by the acquisition of CD1a. ChIP assays confirmed the binding of ICN1 to the DTX1, NOTCH3 and IL7R enhancers in T-cell precursors. Consistent with the Q-RT-PCR analysis, GSI treatment impaired RNAPII recruitment to Notch-target genes. Importantly, several components of the newly characterized Notch-activation complex, including BRG1, PHF8 and LSD1 were associated with ICN1-containing enhancers. As in T-ALL cells, binding of BRG1 and PHF8, but not LSD1 requires activation of Notch signaling. Furthermore, we detected an accumulation of the repressive marks H3K9me2 and H3K27me2 after turning off Notch signaling. This is consistent with LSD1 and PHF8 demethylase activities. These results suggest that ICN1 might trigger the formation of a similar activation complex in developing T-cell and their malignant counterpart.

### DISCUSSION

Notch signaling is involved in virtually all developmental processes and implicated in many human diseases including T-cell lymphoblastic leukemia. Here the inventors identified nuclear ICN1-partners in human T-cell leukemia providing a framework to elucidate ICN1 regulation and mechanisms of action.

Insights into the Molecular Mechanisms Involved in Notch-Dependent Transcription:

Upon Notch activation, ICN1 directs the formation of the ternary ICN1-CSL-MAML1 complex required for Notch transcriptional responses. Here the inventors expand the understanding of Notch-mediated transcription by identifying new components of the Notch-activation complex. An important finding from this study is that Notch activation leads to the assembly of a large multisubunit complex containing ICN1-CSL-MAML1 and several classes of transcriptional regulators that could act at different steps of the transcriptional activation process. The control of gene expression through chromatin requires proteins that enzymatically regulate nucleosomal structure and histone modifications. The SWI/SNF remodeling complex PBAF was found here to interact with the core Notch-activation complex. Importantly, the catalytic subunit of PBAF, BRG1, is required for endogenous Notch-target gene expression in T-ALL cells. This finding is consistent with the previously reported role of BRG1 in Notch signaling during mouse embryonic development (Takeuchi et al., 2007). These data also uncovers an important function for AF4p12 in Notch-mediated transcriptional activation. AF4p12 is a poorly characterized protein that was first identified as one of the MLL translocation partners in leukemia (Hayette et al., 2005). Importantly, its C-terminal part displays transcription activation properties when fused to GAL4 DNA-binding domain (Hayette et al., 2005). While the precise role of AF4p12 awaits further investigation, our results indicate that it is required for RNAPII recruitment at several Notch-target genes. AF4p12 therefore acts as a transcriptional coactivator of ICN1.

Epigenetic regulation of Notch-target genes:

The results provide strong evidence indicating that histone modifications play a central role in Notch-target gene regulation. Several histone modifiers, including the BRE1 subunit RNF40, and the demethylases LSD1 and PHF8, were detected by MS. Interestingly, the homologue of BRE1 is required for Notch activity in drosophila (Bray et al., 2005), suggesting that H2B monoubiquitination might play a conserved function in Notch signaling. In the current study, the inventors focused on the role of LSD1 and PHF8 in modulating Notch responses.

Previous studies in various species have linked LSD1 to the repression of Notch-target genes (Di Stefano et al., 2011; Mulligan et al., 2011; Wang et al., 2007). In agreement with these observations, we found that LSD1 is bound to chromatin as part of the CSL-repressor complex and prevents transcription by maintaining low levels of H3K4me2. However, our study reveals a transcriptional coactivator function for LSD1 in the context of the Notch-activation complex. Indeed, we show that LSD1 is required for Notch-mediated activation of its target genes by insuring efficient H3K9me2 demethylation. Consistently, mutant alleles of drosophila LSD1 suppressed gain-of-function phenotypes of Notch (Di Stefano et al., 2011), suggesting a conserved role of LSD1 in the activation of Notch signaling. What are the mechanisms that govern alteration of LSD1 activity after Notch activation? First, LSD1 substrate specificity could be modulated as a result of CSL complex remodeling. Second, histone tail modifications may play a role. Accordingly, in vitro demethylation of H3K4 by LSD1 is completely blocked by Ser10 phosphorylation (Forneris et al., 2005), a mark that is strongly increased after ICN1 recruitment (Fryer et al., 2004). Thus, as a consequence of Notch activation, a sequence of events that remains to be determined, including remodeling of proteins complexes and histone marks, might modify LSD1 catalytic activity.

Similarly to LSD1, PHD finger- and JmjC-containing histone demethylase, PHF8, exhibits differential substrate specificities in different contexts. For example, PHD-mediated binding to adjacent H3K4me3 is required for H3K9me1/2 but not H3K27me2 demethylation (Horton et al., 2010; Liu et al., 2010b). In the context of Notch responses, PHF8 recruitment to ICN1-containing enhancers is associated with a robust demethylation of H3K27me2. Our results also reveal an additional activity of PHF8 toward lysine 9 at the TSS region, probably mediated through its interaction with H3K4me3. Because most Notch-responsive genes lack ICN1 binding sites at the promoter region and are regulated through enhancers (containing low levels of H3K4me3) (Wang et al., 2011), the major PHF8 substrate is likely H3K27me2. Thus, ICN1 recruitment to Notch-responsive enhancers is associated with at least two demethylase activities: H3K9me1/me2 demethylation by LSD1 and H3K27me2 by PHF8. However, in some cases, both LSD1 and PHF8 might contribute to Notch-induced demethylation of H3K9me1/2. Additionally, PHF8 and LSD1 may function by targeting other substrates, including non-histone proteins.

Additional Partners Involved in Notch Transcriptional Activity?

Other ICN1-interacting proteins identified in this study may also associate with the Notch-activation complex and play non-redundant functions in Notch-target genes activation (Table 1). Some of these factors, such as the corepressor/coactivator exchange factor TBLR1 or the poorly characterized nuclear protein ERH, have been described as positive regulators of Notch signaling. Thus, further characterization of additional partners will undoubtedly help to complete our understanding of Notch functions. Moreover, factors involved in transcriptional repression, including the NuRD complex and subunits of the polycomb repressive complex 1 (PRC1), copurified with ICN1. Since the primary function of ICN1 is to activate transcription, investigations on the biological significance of these interactions might reveal novel aspects of Notch signaling.

### REFERENCES

Throughout this application, various references describe the state of the art to which this invention pertains. The disclosures of these references are hereby incorporated by reference into the present disclosure.
Abramson, J., Giraud, M., Benoist, C., and Mathis, D. (2010). Aire's partners in the molecular control of immunological tolerance. Cell 140, 123-135.
Agoulnik, I. U., Bingman, W. E., 3rd, Nakka, M., Li, W., Wang, Q., Liu, X. S., Brown, M., and Weigel, N. L. (2008). Target gene-specific regulation of androgen receptor activity by p42/p44 mitogen-activated protein kinase. Mol Endocrinol 22, 2420-2432.
Aifantis, I., Raetz, E., and Buonamici, S. (2008). Molecular pathogenesis of T-cell leukaemia and lymphoma. Nat Rev Immunol 8, 380-390.
Andersson, E. R., Sandberg, R., and Lendahl, U. (2011). Notch signaling: simplicity in design, versatility in function. Development 138, 3593-3612.
Andreu-Agullo, C., Maurin, T., Thompson, C. B., and Lai, E. C. (2012). Ars2 maintains neural stem-cell identity through direct transcriptional activation of Sox2. Nature 481, 195-198.
Artavanis-Tsakonas, S., Rand, M. D., and Lake, R. J. (1999). Notch signaling: cell fate control and signal integration in development. Science 284, 770-776.
Aste-Amézaga M, Zhang N, Lineberger J E, et al. Characterization of Notch1 antibodies that inhibit signaling of both normal and mutated Notch1 receptors; PLoS One. 2010 Feb. 8; 5(2):e9094.
Borggrefe, T., and Oswald, F. (2009). The Notch signaling pathway: transcriptional regulation at Notch target genes. Cell Mol Life Sci 66, 1631-1646.
Bray, S., Musisi, H., and Bienz, M. (2005). Bre1 is required for Notch signaling and histone modification. Dev Cell 8, 279-286.
Bungard, D., Fuerth, B. J., Zeng, P. Y., Faubert, B., Maas, N. L., Viollet, B., Carling, D., Thompson, C. B., Jones, R. G., and Berger, S. L. (2010). Signaling kinase AMPK activates stress-promoted transcription via histone H2B phosphorylation. Science 329, 1201-1205.
Chanarat, S., Seizl, M., and Strasser, K. (2011). The Prpl9 complex is a novel transcription elongation factor required for TREX occupancy at transcribed genes. Genes Dev 25, 1147-1158.
Chen, Y. J., Wang, Y. N., and Chang, W. C. (2007). ERK2-mediated C-terminal serine phosphorylation of p300 is vital to the regulation of epidermal growth factor-induced keratin 16 gene expression. J Biol Chem 282, 27215-27228.
Core, L. J., Waterfall, J. J., and Lis, J. T. (2008). Nascent RNA sequencing reveals widespread pausing and divergent initiation at human promoters. Science 322, 1845-1848.
Dango, S., Mosammaparast, N., Sowa, M. E., Xiong, L. J., Wu, F., Park, K., Rubin, M., Gygi, S., Harper, J. W., and Shi, Y. (2011). DNA unwinding by ASCC3 helicase is coupled to ALKBH3-dependent DNA alkylation repair and cancer cell proliferation. Mol Cell 44, 373-384.
Di Stefano, L., Walker, J. A., Burgio, G., Corona, D. F., Mulligan, P., Naar, A. M., and Dyson, N. J. (2011). Functional antagonism between histone H3K4 demethylases in vivo. Genes Dev 25, 17-28.
Fay, A., Misulovin, Z., Li, J., Schaaf, C. A., Gause, M., Gilmour, D. S., and Dorsett, D. (2011). Cohesin selectively binds and regulates genes with paused RNA polymerase. Curr Biol 21, 1624-1634.
Feng, W., Yonezawa, M., Ye, J., Jenuwein, T., and Grummt, I. (2010). PHF8 activates transcription of rRNA genes through H3K4me3 binding and H3K9me1/2 demethylation. Nat Struct Mol Biol 17, 445-450.
Forneris, F., Binda, C., Vanoni, M. A., Battaglioli, E., and Mattevi, A. (2005). Human histone demethylase LSD1 reads the histone code. J Biol Chem 280, 41360-41365.
Fortschegger, K., de Graaf, P., Outchkourov, N. S., van Schalk, F. M., Timmers, H. T., and Shiekhattar, R. (2010). PHF8 targets histone methylation and RNA polymerase II to activate transcription. Mol Cell Biol 30, 3286-3298.
Fryer, C. J., White, J. B., and Jones, K. A. (2004). Mastermind recruits CycC:CDK8 to phosphorylate the Notch ICD and coordinate activation with turnover. Mol Cell 16, 509-520.
Garcia-Bassets, I., Kwon, Y. S., Telese, F., Prefontaine, G. G., Hutt, K. R., Cheng, C. S., Ju, B. G., Ohgi, K. A., Wang, J., Escoubet-Lozach, L., et al. (2007). Histone methylation-dependent mechanisms impose ligand dependency for gene activation by nuclear receptors. Cell 128, 505-518.
Gonzalez-Garcia, S., Garcia-Peydro, M., Martin-Gayo, E., Ballestar, E., Esteller, M., Bornstein, R., de la Pompa, J. L., Ferrando, A. A., and Toribio, M. L. (2009). CSL-MAML-dependent Notch1 signaling controls T lineage-specific IL-7R{alpha} gene expression in early human thymopoiesis and leukemia. J Exp Med 206, 779-791.
Haffner, M. C., Aryee, M. J., Toubaji, A., Esopi, D. M., Albadine, R., Gurel, B., Isaacs, W. B., Bova, G. S., Liu, W., Xu, J., et al. (2010). Androgen-induced TOP2B-mediated double-strand breaks and prostate cancer gene rearrangements. Nat Genet 42, 668-675.
Harada, N., Yokoyama, T., Yamaji, R., Nakano, Y., and Inui, H. (2008). RanBP10 acts as a novel coactivator for the androgen receptor. Biochem Biophys Res Commun 368, 121-125.
Hayette, S., Cornillet-Lefebvre, P., Tigaud, I., Struski, S., Forissier, S., Berchet, A., Doll, D., Gillot, L., Brahim, W., Delabesse, E., et al. (2005). AF4p12, a human homologue to the furry gene of Drosophila, as a novel MLL fusion partner. Cancer Res 65, 6521-6525.
Ho, L., and Crabtree, G. R. (2010). Chromatin remodelling during development. Nature 463, 474-484.
Horton, J. R., Upadhyay, A. K., Qi, H. H., Zhang, X., Shi, Y., and Cheng, X. (2010). Enzymatic and structural insights for substrate specificity of a family of jumonji histone lysine demethylases. Nat Struct Mol Biol 17, 38-43.
Ju, B. G., Lunyak, V. V., Perissi, V., Garcia-Bassets, I., Rose, D. W., Glass, C. K., and Rosenfeld, M. G. (2006). A topoisomerase IIbeta-mediated dsDNA break required for regulated transcription. Science 312, 1798-1802.
Jung, D. J., Sung, H. S., Goo, Y. W., Lee, H. M., Park, O. K., Jung, S. Y., Lim, J., Kim, H. J., Lee, S. K., Kim, T. S., et al. (2002). Novel transcription coactivator complex containing activating signal cointegrator 1. Mol Cell Biol 22, 5203-5211.
Kagey, M. H., Newman, J. J., Bilodeau, S., Zhan, Y., Orlando, D. A., van Berkum, N. L., Ebmeier, C. C., Goossens, J., Rahl, P. B., Levine, S. S., et al. (2010). Mediator and cohesin connect gene expression and chromatin architecture. Nature 467, 430-435.
Kleine-Kohlbrecher, D., Christensen, J., Vandamme, J., Abarrategui, I., Bak, M., Tommerup, N., Shi, X., Gozani, O., Rappsilber, J., Salcini, A. E., et al. (2010). A functional link between the histone demethylase PHF8 and the transcription factor ZNF711 in X-linked mental retardation. Mol Cell 38, 165-178.
Kopan R, Ilagan M X; The canonical Notch signaling pathway: unfolding the activation mechanism; Cell. 2009 Apr. 17; 137(2):216-33.
Kristie, T. M., and Sharp, P. A. (1993). Purification of the cellular C1 factor required for the stable recognition of the Oct-1 homeodomain by the herpes simplex virus alpha-trans-induction factor (VP16). J Biol Chem 268, 6525-6534.
Kundu, M., Compton, S., Garrett-Beal, L., Stacy, T., Starost, M. F., Eckhaus, M., Speck, N. A., and Liu, P. P. (2005). Runx1 deficiency predisposes mice to T-lymphoblastic lymphoma. Blood 106, 3621-3624.
Li, X. Y., and Green, M. R. (1998). The HIV-1 Tat cellular coactivator Tat-SF1 is a general transcription elongation factor. Genes Dev 12, 2992-2996.
Liu, C., Huang, W., and Lei, Q. (2011). Regulation and function of the TAZ transcription co-activator. Int J Biochem Mol Biol 2, 247-256.
Liu, H., Chi, A. W., Arnett, K. L., Chiang, M. Y., Xu, L., Shestova, O., Wang, H., Li, Y. M., Bhandoola, A., Aster, J. C., et al. (2010a). Notch dimerization is required for leukemogenesis and T-cell development. Genes Dev 24, 2395-2407.
Liu, W., Tanasa, B., Tyurina, O. V., Zhou, T. Y., Gassmann, R., Liu, W. T., Ohgi, K. A., Benner, C., Garcia-Bassets, I., Aggarwal, A. K., et al. (2010b). PHF8 mediates histone H4 lysine 20 demethylation events involved in cell cycle progression. Nature 466, 508-512.
Loenarz, C., Ge, W., Coleman, M. L., Rose, N. R., Cooper, C. D., Klose, R. J., Ratcliffe, P. J., and Schofield, C. J. (2010). PHF8, a gene associated with cleft lip/palate and mental retardation, encodes for an Nepsilon-dimethyl lysine demethylase. Hum Mol Genet 19, 217-222.
Madak-Erdogan, Z., Lupien, M., Stossi, F., Brown, M., and Katzenellenbogen, B. S. (2011). Genomic collaboration of estrogen receptor alpha and extracellular signal-regulated kinase 2 in regulating gene and proliferation programs. Mol Cell Biol 31, 226-236.
Malik, S., and Roeder, R. G. (2005). Dynamic regulation of pol II transcription by the mammalian Mediator complex. Trends Biochem Sci 30, 256-263.
Magri, M., Yatim, A., Benne, C., Balbo, M., Henry, A., Serraf, A., Sakano, S., Gazzolo, L., Levy, Y., and Lelievre, J. D. (2009). Notch ligands potentiate IL-7-driven proliferation and survival of human thymocyte precursors. Eur J Immunol 39, 1231-1240.
Metzger, E., Wissmann, M., Yin, N., Muller, J. M., Schneider, R., Peters, A. H., Gunther, T., Buettner, R., and Schule, R. (2005). LSD1 demethylates repressive histone marks to promote androgen-receptor-dependent transcription. Nature 437, 436-439.
Miele L, Miao H, Nickoloff B J; Notch signaling as a novel cancer therapeutic target; Curr Cancer Drug Targets. 2006 June; 6(4):313-23.
Mulligan, P., Yang, F., Di Stefano, L., Ji, J. Y., Ouyang, J., Nishikawa, J. L., Toiber, D., Kulkarni, M., Wang, Q., Najafi-Shoushtari, S. H., et al. (2011). A SIRT1-LSD1 corepressor complex regulates Notch target gene expression and development. Mol Cell 42, 689-699.
Nam, Y., Sliz, P., Song, L., Aster, J. C., and Blacklow, S. C. (2006). Structural basis for cooperativity in recruitment of MAML coactivators to Notch transcription complexes. Cell 124, 973-983.
Nam, Y., Sliz, P., Pear, W. S., Aster, J. C., and Blacklow, S. C. (2007). Cooperative assembly of higher-order Notch complexes functions as a switch to induce transcription. Proc Natl Acad Sci USA 104, 2103-2108.
Nock, A., Ascano, J. M., Jones, T., Barrero, M. J., Sugiyama, N., Tomita, M., Ishihama, Y., and Malik, S. (2009). Identification of DNA-dependent protein kinase as a cofactor for the forkhead transcription factor FoxA2. J Biol Chem 284, 19915-19926.
Osley, M. A. (2006). Regulation of histone H2A and H2B ubiquitylation. Brief Funct Genomic Proteomic 5, 179-189.
Qi, H. H., Sarkissian, M., Hu, G. Q., Wang, Z., Bhattacharjee, A., Gordon, D. B., Gonzales, M., Lan, F., Ongusaha, P. P., Huarte, M., et al. (2010). Histone H4K20/H3K9 demethylase PHF8 regulates zebrafish brain and craniofacial development. Nature 466, 503-507.
Qiu, J., Shi, G., Jia, Y., Li, J., Wu, M., Dong, S., and Wong, J. (2010). The X-linked mental retardation gene PHF8 is a histone demethylase involved in neuronal differentiation. Cell Res 20, 908-918.
Pauli, A., van Bemmel, J. G., Oliveira, R. A., Itoh, T., Shirahige, K., van Steensel, B., and Nasmyth, K. (2010). A direct role for cohesin in gene regulation and ecdysone response in Drosophila salivary glands. Curr Biol 20, 1787-1798.
Perez-Gonzalez, A., Rodriguez, A., Huarte, M., Salanueva, I. J., and Nieto, A. (2006). hCLE/CGI-99, a human protein that interacts with the influenza virus polymerase, is a mRNA transcription modulator. J Mol Biol 362, 887-900.
Perillo, B., Ombra, M. N., Bertoni, A., Cuozzo, C., Sacchetti, S., Sasso, A., Chiariotti, L., Malorni, A., Abbondanza, C., and Avvedimento, E. V. (2008). DNA oxidation as triggered by H3K9me2 demethylation drives estrogen-induced gene expression. Science 319, 202-206.
Perissi, V., Aggarwal, A., Glass, C. K., Rose, D. W., and Rosenfeld, M. G. (2004). A corepressor/coactivator exchange complex required for transcriptional activation by nuclear receptors and other regulated transcription factors. Cell 116, 511-526.
Perissi, V., Scafoglio, C., Zhang, J., Ohgi, K. A., Rose, D. W., Glass, C. K., and Rosenfeld, M. G. (2008). TBL1 and TBLR1 phosphorylation on regulated gene promoters overcomes dual CtBP and NCoR/SMRT transcriptional repression checkpoints. Mol Cell 29, 755-766.
Poirier, M. B., Laflamme, L., and Langlois, M. F. (2006). Identification and characterization of RanBPM, a novel coactivator of thyroid hormone receptors. J Mol Endocrinol 36, 313-325.
Rao, M. A., Cheng, H., Quayle, A. N., Nishitani, H., Nelson, C. C., and Rennie, P. S. (2002). RanBPM, a nuclear protein that interacts with and regulates transcriptional activity of androgen receptor and glucocorticoid receptor. J Biol Chem 277, 48020-48027.
Rothenberg, E. V., and Taghon, T. (2005). Molecular genetics of T cell development. Annu Rev Immunol 23, 601-649.
Sakane, N., Kwon, H. S., Pagans, S., Kaehlcke, K., Mizusawa, Y., Kamada, M., Lassen, K. G., Chan, J., Greene, W. C., Schnoelzer, M., et al. (2011). Activation of HIV transcription by the viral Tat protein requires a demethylation step mediated by lysine-specific demethylase 1 (LSD1/KDM1). PLoS Pathog 7, e1002184.
Schmitt, T. M., and Zuniga-Pflucker, J. C. (2002). Induction of T cell development from hematopoietic progenitor cells by delta-like-1 in vitro. Immunity 17, 749-756.
Seitan, V. C., Hao, B., Tachibana-Konwalski, K., Lavagnolli, T., Mira-Bontenbal, H., Brown, K. E., Teng, G., Carroll, T., Terry, A., Horan, K., et al. (2011). A role for cohesin in T-cell-receptor rearrangement and thymocyte differentiation. Nature 476, 467-471.
Shi, Y., Lan, F., Matson, C., Mulligan, P., Whetstine, J. R., Cole, P. A., and Casero, R. A. (2004). Histone demethylation mediated by the nuclear amine oxidase homolog LSD1. Cell 119, 941-953.
Snyder, M., He, W., and Zhang, J. J. (2005). The DNA replication factor MCM5 is essential for Stat1-mediated transcriptional activation. Proc Natl Acad Sci USA 102, 14539-14544.
South, A. P., Cho, R. J., and Aster, J. C. (2012). The double-edged sword of Notch signaling in cancer. Semin Cell Dev Biol.
Takeuchi, J. K., Lickert, H., Bisgrove, B. W., Sun, X., Yamamoto, M., Chawengsaksophak, K., Hamada, H., Yost, H. J., Rossant, J., and Bruneau, B. G. (2007). Baf60c is a nuclear Notch signaling component required for the establishment of left-right asymmetry. Proc Natl Acad Sci USA 104, 846-851.
Tyagi, S., Ochem, A., and Tyagi, M. (2011). DNA-dependent protein kinase interacts functionally with the RNA polymerase II complex recruited at the human immunodeficiency virus (HIV) long terminal repeat and plays an important role in HIV gene expression. J Gen Virol 92, 1710-1720.
van der Knaap, J. A., Kumar, B. R., Moshkin, Y. M., Langenberg, K., Krijgsveld, J., Heck, A. J., Karch, F., and Verrijzer, C. P. (2005). GMP synthetase stimulates histone H2B deubiquitylation by the epigenetic silencer USP7. Mol Cell 17, 695-707.
Vicent, G. P., Ballare, C., Nacht, A. S., Clausell, J., Subtil-Rodriguez, A., Quiles, I., Jordan, A., and Beato, M. (2006). Induction of progesterone target genes requires activation of Erk and Msk kinases and phosphorylation of histone H3. Mol Cell 24, 367-381.
Vogel, J. L., and Kristie, T. M. (2000). The novel coactivator C1 (HCF) coordinates multiprotein enhancer formation and mediates transcription activation by GABP. EMBO J 19, 683-690.
Wakabayashi, Y., Inoue, J., Takahashi, Y., Matsuki, A., Kosugi-Okano, H., Shinbo, T., Mishima, Y., Niwa, O., and Kominami, R. (2003). Homozygous deletions and point mutations of the Rit1/Bcl11b gene in gamma-ray induced mouse thymic lymphomas. Biochem Biophys Res Commun 301, 598-603.
Wan, C., Tempel, W., Liu, Z. J., Wang, B. C., and Rose, R. B. (2005). Structure of the conserved transcriptional repressor enhancer of rudimentary homolog. Biochemistry 44, 5017-5023.
Wang, H., Zou, J., Zhao, B., Johannsen, E., Ashworth, T., Wong, H., Pear, W. S., Schug, J., Blacklow, S. C., Arnett, K. L., et al. (2011). Genome-wide analysis reveals conserved and divergent features of Notch1/RBPJ binding in human and murine T-lymphoblastic leukemia cells. Proc Natl Acad Sci USA 108, 14908-14913.
Wang, J., Scully, K., Zhu, X., Cai, L., Zhang, J., Prefontaine, G. G., Krones, A., Ohgi, K. A., Zhu, P., Garcia-Bassets, I., et al. (2007). Opposing LSD1 complexes function in developmental gene activation and repression programmes. Nature 446, 882-887.
Watanabe, M., Yanagisawa, J., Kitagawa, H., Takeyama, K., Ogawa, S., Arao, Y., Suzawa, M., Kobayashi, Y., Yano, T., Yoshikawa, H., et al. (2001). A subfamily of RNA-binding DEAD-box proteins acts as an estrogen receptor alpha coactivator through the N-terminal activation domain (AF-1) with an RNA coactivator, SRA. EMBO J 20, 1341-1352.
Weng, A. P., Ferrando, A. A., Lee, W., Morris, J. P. t., Silverman, L. B., Sanchez-Irizarry, C., Blacklow, S. C., Look, A. T., and Aster, J. C. (2004). Activating mutations of NOTCH1 in human T cell acute lymphoblastic leukemia. Science 306, 269-271.
Wilson, J. J., and Kovall, R. A. (2006). Crystal structure of the CSL-Notch-Mastermind ternary complex bound to DNA. Cell 124, 985-996.
Wong, R. H., Chang, I., Hudak, C. S., Hyun, S., Kwan, H. Y., and Sul, H. S. (2009). A role of DNA-PK for the metabolic gene regulation in response to insulin. Cell 136, 1056-1072.
Wortham, N. C., Ahamed, E., Nicol, S. M., Thomas, R. S., Periyasamy, M., Jiang, J., Ochocka, A. M., Shousha, S., Huson, L., Bray, S. E., et al. (2009). The DEAD-box protein p72 regulates ERalpha-/oestrogen-dependent transcription and cell growth, and is associated with improved survival in ERalpha-positive breast cancer. Oncogene 28, 4053-4064.
Wu, X., Li, H., and Chen, J. D. (2001). The human homologue of the yeast DNA repair and TFIIH regulator MMS19 is an AF-1-specific coactivator of estrogen receptor. J Biol Chem 276, 23962-23968.
Wu Y, Cain-Hom C, Choy L, et al.; Therapeutic antibody targeting of individual Notch receptors. Nature. 2010 Apr. 15; 464(7291):1052-7. Xiang, J., Ouyang, Y., Cui, Y., Lin, F., Ren, J., Long, M., Chen, X., Wei, J., and Zhang, H. (2011). Silencing of Notch3 Using shRNA Driven by Survivin Promoter Inhibits Growth and Promotes Apoptosis of Human T-Cell Acute Lymphoblastic Leukemia Cells. Clin Lymphoma Myeloma Leuk.
Zhang, J., Ding, L., Holmfeldt, L., Wu, G., Heatley, S. L., Payne-Turner, D., Easton, J., Chen, X., Wang, J., Rusch, M., et al. (2012). The genetic basis of early T-cell precursor acute lymphoblastic leukaemia. Nature 481, 157-163.
Zhang, J. J., Zhao, Y., Chait, B. T., Lathem, W. W., Ritzi, M., Knippers, R., and Darnell, J. E., Jr. (1998). Ser727-dependent recruitment of MCM5 by Stat1alpha in IFN-gamma-induced transcriptional activation. EMBO J 17, 6963-6971.
Zhou, Q., and Sharp, P. A. (1996). Tat-SF1: cofactor for stimulation of transcriptional elongation by HIV-1 Tat. Science 274, 605-610.
Zhu, B., Zheng, Y., Pham, A. D., Mandal, S. S., Erdjument-Bromage, H., Tempst, P., and Reinberg, D. (2005). Monoubiquitination of human histone H2B: the factors involved and their roles in HOX gene regulation. Mol Cell 20, 601-611.
Zhu, Z., Wang, Y., Li, X., Xu, L., Wang, X., Sun, T., Dong, X., Chen, L., Mao, H., Yu, Y., et al. (2010). PHF8 is a histone H3K9me2 demethylase regulating rRNA synthesis. Cell Res 20, 794-801.

1. A method for screening a compound capable of inhibiting the Notch1 transcriptional activity, comprising:
(a) identifying a compound that inhibits the specific interaction of intracellular domain of NOTCH1 (ICN1) with a nuclear protein required for Notch1 transcriptional activity as depicted in Table 1, or
(b) identifying a compound that inhibits the expression of a nuclear protein as depicted in Table 1, or
(c) identifying a compound that inhibits the activity of a nuclear protein as depicted in Table 1.

2. A method for the prevention or treatment of cell proliferative diseases and disorders associated with overexpression and/or activation of Notch1 in a subject in need thereof comprising administering to said subject a therapeutically effective amount of a compound that inhibits the interaction between ICN1 and a nuclear protein required for Notch1 transcriptional activity as depicted in Table 1.

3. The method according to claim 2, wherein the nuclear protein is selected in the group consisting of PHF8, AF4p12, LSD1 and BRG1.

4. The method according to claim 3, wherein said compound is an inhibitor of PHF8, AF4p12, LSD1 or BRG1 gene expression.

5. A method for the prevention or treatment of cell proliferative diseases and disorders associated with overexpression and/or activation of Notch1 in a subject in need thereof comprising administering to said subject a therapeutically effective amount of a compound that inhibits the activity of PHF8 or AF4p12.

6. The method according to claim 2, wherein the cell proliferative disease and disorder associated with overexpression and/or activation of Notch1 is selected from the group consisting of breast cancer, ovarian cancer, prostate cancer, cervical cancer, lung cancer, brain cancers, melanomas, gastrointestinal cancers, head and neck cancer, and hematopoietic cell cancers.

7. The method according to claim 6, wherein the hematopoietic cell cancer is T-cell acute lymphoblastic leukemia (T-ALL).

8. The method according to claim 5, wherein the cell proliferative disease and disorder associated with overexpression and/or activation of Notch1 is selected from the group consisting of breast cancer, ovarian cancer, prostate cancer, cervical cancer, lung cancer, brain cancers, melanomas, gastrointestinal cancers, head and neck cancer, and hematopoietic cell cancers.

9. The method according to claim 8, wherein the hematopoietic cell cancer is T-cell acute lymphoblastic leukemia (T-ALL).
